(19) United States
(12) Patent Application Publication
(10) Pub. No.: US 2026/0063419 A1
(43) Pub. Date: Mar. 5, 2026

(54) WATER BODY MONITORING AND/OR AUTOMATIC CONTROL THROUGH A HIGH-PRECISION SENSOR BUOY

(71) Applicant: Leon L Wagler, Angola, IN (US)

(72) Inventors: Leon L Wagler, Hudson, IN (US); RONALD EICHER, Hicksville, OH (US)

(21) Appl. No.: 19/311,514

(22) Filed: Aug. 27, 2025

Related U.S. Application Data
(60) Provisional application No. 63/732,555, filed on Aug. 27, 2024.

(51) Int. Cl. G01C 13/00; B63B 22/00; G01N 33/18

(52) U.S. Cl. CPC G01C 13/008; B63B 22/00; G01N 33/1886; B63B 2022/006; B63B 2211/02

(57) ABSTRACT

A water level monitoring system includes a sensor buoy with a GPS unit and RTK correction system for high-precision elevation measurements in bodies of water. The sensor buoy is easily deployable in existing facilities and can be used to survey bodies of water during site setup. The sensor buoy includes a float, geospatial positioning unit, wireless network interface, and/or power source such as a solar array. A coordination server may receive geospatial coordinates from the sensor buoy including elevation data and use site-specific depth and volume functions to calculate water levels. Automated alerts may be generated when water levels exceed thresholds and/or control infrastructure devices such as valves and pumps to prevent overflow conditions and improve operational efficiency. The sensor buoy may operates autonomously with adaptive data resolution, accuracy, and/or precision adjustment based on water level conditions and power management needs. Applications include monitoring and controlling wastewater lagoons, reservoirs, and/or agricultural operations.

### CLAIM FOR PRIORITY AND CROSS REFERENCE TO RELATED APPLICATIONS

This patent application claims priority from, and hereby incorporates by reference: U.S. provisional patent application No. 63/732,555, titled ‘Intelli Buoy’, filed Aug. 27, 2024.

### FIELD OF TECHNOLOGY

This disclosure relates generally to data processing devices and, more particularly, to a method, a device, and/or a system of water body monitoring and/or automatic control through a high-precision sensor buoy.

### BACKGROUND

Water level monitoring in bodies of water such as reservoirs, wastewater lagoons, mining lagoons, and treatment facilities, presents significant challenges for operational efficiency and/or environmental compliance. Traditional water level measurement methods may rely on manual inspection, visual estimates, and/or fixed gauge systems that may provide limited data resolution, require significant equipment or permanent installations, and/or require frequent maintenance visits. These conventional approaches may pose challenges operational efficiency and/or environmental monitoring requirements, which demand affordable, continuous, precise, and/or remotely accessible water level data.

Existing water level monitoring systems may have several technical limitations. Manual measurement systems may be labor-intensive and provide only periodic data for water levels, making it difficult to detect rapid changes or predict overflow conditions. More detailed depth monitoring meters may include multiple mechanical parts that might be prone to degradation or failure over time. Fixed gauge systems, while providing continuous monitoring, are sometimes limited by their installation requirements and susceptibility to environmental damage, especially in saline or corrosive liquids common to remediation lagoons. Additionally, some current systems may lack the precision necessary for accurate and/or precise volume calculations in shallow, wide lagoons and/or irregularly shaped bodies of water such as reservoirs. Related to these challenges, it may also be difficult to predict the capacity of a body of water, especially in light of changing usage, climate, and/or weather events.

The need for precise water level monitoring may have become increasingly important due to the need for careful water management, stricter environmental regulations, and the growing emphasis on preventing overflow events that can result in wasted water storage, damage to dams or lagoons, and/or environmental contamination. Wastewater treatment facilities, agricultural operations, and/or industrial sites require reliable monitoring systems that can provide early warning of potential overflow conditions while also supporting operational efficiency and compliance reporting requirements. However, many such systems are expensive, difficult or time consuming to build or deploy, and/or have limited specialized uses.

Furthermore, existing monitoring systems may operate as standalone devices without integration capabilities for broader operational and/or environmental management systems. This limitation may prevent operators from implementing automated responses to changing water levels or incorporating water level data into predictive models that could optimize facility operations and prevent environmental incidents.

There exists a need for improved water level monitoring systems, devices, and/or methods that are fast and easy to deploy, affordable, flexible, configurable, high accuracy, high-precision, operate autonomously with minimal maintenance requirements, and/or integrate with broader operation and environmental management systems to enable predictive monitoring, alert, and/or automated response capabilities.

### SUMMARY

In one embodiment, a sensor buoy for monitoring water level of a body of water includes a float for floating the sensor buoy on a surface of the body of water, an elevation rod coupled to the float, and a geospatial positioning unit coupled to the elevation rod and electrically coupled to a power source. The power source includes a battery coupled to the sensor buoy and the geospatial positioning unit includes a GPS unit and a spatial correction chip comprising an RTK unit. The sensor buoy further includes an energy generator coupled to the sensor buoy. The energy generator includes a solar panel.

The sensor buoy also includes a wireless network interface controller configured to communicatively couple to a server through a wireless network, a processor, and a computer readable media that is non-transitory. The computer readable media includes computer readable instructions that when executed: determine a first geospatial coordinate and a first precision value from the geospatial positioning unit; receive a correction data from the spatial correction chip; generate a corrected geospatial coordinate; and transmit the corrected geospatial coordinate to the server over the wireless network. The sensor buoy also includes a tether coupled to the float and an anchor coupled to the tether for anchoring the sensor buoy to an anchor point on a floor of the body of water.

The geospatial positioning unit may be coupled to the elevation rod at a first end of the elevation rod and the elevation rod may coupled to the float at a second end of the elevation rod. The sensor buoy may further include a rod coupler configured to detachably couple the elevation rod to the float at the first end of the elevation rod such that the elevation rod usable as a survey rod to easily gather a site data for the body of water.

The computer readable media may further include computer readable instructions that when executed: receive a request to initiate a site acquisition mode to gather a site data for the body of water; configure (i) a continuous point acquisition mode determining geospatial coordinates at a first coordinate determination rate at least as fast as one point per ten seconds and/or (ii) a manual point acquisition mode; initiate a site data object for the body of water; gather the site data comprising a first set of geospatial coordinates, each geospatial coordinate of the first set of geospatial coordinates paired with a precision value; receive a request to end the site acquisition mode gathering the site data for the body of water; commit the site data to the site data object; and transmit the site data object to the server over the wireless network.

The computer readable media may further include computer readable instructions that when executed: receive a reduced data quality request in response to a drop in a depth of the body of water, and configure a second coordinate determination rate that at lest one of (i) slows the rate at which geospatial coordinates are determined to increase an energy efficiency of the power source; and (ii) reduces a quantity of geospatial coordinates gathered for calculating average geospatial coordinates.

The computer readable media may further include computer readable instructions that when executed: set a timer; initiate a low power mode; determine expiration of the up timer; initiate an active mode; and determine the first geospatial coordinate and the first precision value from the geospatial positioning unit upon entering the active mode to increase an energy efficiency of the power source.

The sensor buoy may also include a ballast coupled to the float to weight the sensor buoy such that the elevation rod remains upright when the sensor buoy floats on the body of water. An environmental sensor include a humidity sensor, a temperature sensor, and/or a wind sensor can also be included in the sensor buoy. A water quality sensor may be included, such as an oxygenation sensor, a nitrate sensor, a phosphate sensor, a pathogen sensor, and/or a heavy metal sensor. The sensor buoy may also include a backup battery.

The tether may include a corrosion resistant material configured to resist a corrosive chemical in the body of water. For example, the corrosion resistant material include stainless steel and/or an organic polymer.

In another embodiments, a system for monitoring water level in a body of water includes
a network and a sensor buoy communicatively coupled to the network. The sensor buoy includes a geospatial positioning unit coupled to the elevation rod and electrically coupled to a power source, a wireless network interface controller configured to communicatively couple to a server through a wireless network, a processor of the sensor buoy, a computer readable media of the sensor buoy that includes non-transitory comprising computer readable instructions that when executed: determine a first geospatial coordinate comprising an elevation coordinate and a first precision value from the geospatial positioning unit; receive a correction data from the spatial correction chip; generate a corrected geospatial coordinate; and transmit the corrected geospatial coordinate.

The system also includes a server computer including a process of the server computer; and a computer readable media of the server computer that include non-transitory comprising computer readable instructions. The non-transitory comprising computer readable instructions, when executed: (a) receive (i) the first geospatial coordinate and the correction data and/or (ii) the corrected geospatial coordinate from the sensor buoy; (b) query a site profile of the body of water; (c) determine a level of the body of water including (i) inputting the elevation coordinate into a depth function for the wastewater lagoon and determining a depth of wastewater in the wastewater lagoon, and/or (ii) inputting the depth of the wastewater into a volume function of the wastewater lagoon generated based on a contour map of the wastewater lagoon and determining a volume of the wastewater in the wastewater lagoon; (d) determine (i) the depth of wastewater lagoon exceeds a threshold depth, and/or (ii) the volume of the wastewater lagoon exceeds a threshold volume; and (c) generate a potential overflow alert that the wastewater lagoon exceeds at least one of the threshold depth and the volume threshold.

The memory of the sensor buoy may further include computer readable instructions that when executed: receive a request to initiate a site acquisition mode to gather a site data for the body of water; configure (i) a continuous point acquisition mode determining geospatial coordinates at a first coordinate determination rate at least as fast as one point per ten seconds and/or (ii) a manual point acquisition mode; initiate a site data object for the body of water; gather the site data comprising a first set of geospatial coordinates, each geospatial coordinate of the first set of geospatial coordinates paired with a precision value; receive a request to end the site acquisition mode gathering the site data for the body of water; commit the site data to the site data object; and transmit the site data object to the server over the wireless network.

The memory of the sensor buoy may also include computer readable instructions that when executed: receive a reduced data quality requirement request in response to a drop in a depth of the body of water; configure a second coordinate determination rate that (i) slows the rate at which geospatial coordinates are determined to increase an energy efficiency of the power source; and/or (ii) reduces a quantity of geospatial coordinates gathered for calculating average geospatial coordinates; set a timer; initiate a low power mode; determine expiration of the up timer; initiate an active mode; and determine the first geospatial coordinate and the first precision value from the geospatial positioning unit upon entering the active mode to increase an energy efficiency of the power source.

The memory of the server may further include computer readable instructions that when executed: read the precision value upon receipt of the corrected geospatial coordinate; determine the precision value does not meet a precision requirement; delete the precision value; optionally increase a coordinate determination rate of the sensor buoy.

The memory of the server may further include computer readable instructions that when executed: store a water level data comprising at least one of an elevation value of wastewater in the wastewater lagoon over time, a depth of the wastewater in the wastewater lagoon over time, and a volume of the wastewater in the wastewater lagoon over time; generate a level projection for the wastewater lagoon is based on inputs comprising the wastewater level data; determine a date in which a remaining capacity of the wastewater lagoon is exceeded; generate a climate profile comprising average rainfall; associate a precipitation period with an increase in wastewater level; and estimate an increase in the wastewater level based in the climate profile. The level projection for the wastewater lagoon may be based on inputs further including the increase in the wastewater level based on the climate profile.

The memory of the server may further include computer readable instructions that when executed: determine occurrence of a precipitation event; associate the precipitation event with an increase in the wastewater level of the wastewater lagoon; receive weather forecast data; and estimate an increase in the water level based in the weather forecast data. The level projection for the wastewater lagoon may be based on inputs further including the increase in the wastewater level based on the weather forecast data. The memory of the server may further include computer readable instructions that when executed: increase a precision and/or the coordinate determination rate in response to an increase in level of the wastewater to account for increase volume per unit depths as the body of water fills.

The system may further include a second sensor buoy in a second body of water that may be hydrologically coupled and/or hydraulicly coupled to the first body of water. The memory of the server may further include computer readable instructions that when executed: receive a second geospatial coordinate from the second sensor buoy comprising an elevation coordinate of the second geospatial coordinate; determine a wastewater depth of the second wastewater lagoon; determine that the second wastewater lagoon has a remaining capacity to accept discharge from the wastewater lagoon; generate a control instruction comprising at least one of a valve control instruction and a pump control instruction; and transmit the control instruction to at least one of a valve controller and a pump controller through a network to automatically initiate flow of water from the body of water to the second body of water.

The system may further include a device communicatively coupled to the server through the network, the device including a processor of the device and a memory of the device that is a non-transitory computer readable memory comprising a monitoring application comprising computer readable instructions that when executed receive the potential overflow alert to a device comprising a monitoring application. The memory of the server further comprising computer readable instructions that when executed transmit the potential overflow alert to the device. The geospatial positioning unit may include a GPS unit and a spatial correction chip comprising an RTK unit. The first coordinate determination rate may be at least as fast as one point per ten seconds.

According to an aspect of the invention, a method for monitoring water level in a wastewater lagoon comprises generating a first geospatial coordinate at a sensor buoy comprising a geospatial positioning unit wherein the first geospatial coordinate comprises an elevation coordinate, receiving a correction data at the sensor buoy and correcting the first geospatial coordinate with the correction data to generate a corrected geospatial coordinate wherein the corrected geospatial coordinate comprises a precision value following correction by the correction data, querying a lagoon profile of the wastewater lagoon, inputting the elevation coordinate into a depth function for the wastewater lagoon, determining a depth of wastewater in the wastewater lagoon, inputting the depth of the wastewater into a volume function of the wastewater lagoon generated based on a contour map of the wastewater lagoon, determining a volume of the wastewater in the wastewater lagoon, determining at least one of the depth of wastewater lagoon exceeds a threshold depth, and the volume of the wastewater lagoon exceeds a threshold volume, generating a potential overflow alert that the wastewater lagoon exceeds at least one of the threshold depth and the volume threshold, and transmitting the potential overflow alert to a device comprising a monitoring application. This comprehensive monitoring method provides accurate water level determination and immediate alert generation for overflow prevention and environmental compliance.

According to an embodiment, the method further includes receiving a request to initiate a site acquisition mode to gather site data for the wastewater lagoon, configuring a first coordinate determination rate, initiating a site profile for the wastewater lagoon, gathering the site data including a first set of geospatial coordinates collected as the geospatial positioning unit of the sensor buoy travels at least one of in and around the wastewater lagoon, receiving a request to end the site acquisition mode gathering the site data for the wastewater lagoon, receiving the site data at a coordination server, generating a site polygon by bounding the first set of geospatial coordinates, referencing a maximum depth value of the wastewater lagoon, determining a slope specification of the wastewater lagoon, generating at least one of a depth function for the wastewater lagoon and a volume function of the wastewater lagoon, and generating a lagoon profile of the wastewater lagoon and associating at least one of the volume function of the wastewater lagoon and the depth function of the wastewater lagoon.

The method may further include reading the precision value upon receipt of the corrected geospatial coordinate, determining the precision value does not meet a precision requirement, deleting the precision value, and optionally increasing a coordinate determination rate of the sensor buoy. The method may also include storing a wastewater level data that includes an elevation coordinate of wastewater in the wastewater lagoon over time, a depth of the wastewater in the wastewater lagoon over time, and/or a volume of the wastewater in the wastewater lagoon over time, generating a level projection for the wastewater lagoon based on inputs comprising the wastewater level data, determining a date on which a remaining capacity of the wastewater lagoon is exceeded, generating a climate profile comprising average rainfall, associating a precipitation period with an increase in wastewater level, estimating an increase in the wastewater level based on the climate profile wherein the level projection for the wastewater lagoon is based on inputs further comprising the increase in the wastewater level based on the climate profile.

The method may also determine occurrence of a precipitation event, associate the precipitation event with an increase in the wastewater level of the wastewater lagoon, receiving weather forecast data, and estimate an increase in the wastewater level based on the weather forecast data wherein the level projection for the wastewater lagoon is based on inputs further including the increase in the wastewater level based on the weather forecast data.

According to an embodiment, the method may further include generating a control instruction comprising at least one of a valve control instruction and a pump control instruction, and transmitting the control instruction to at least one of a valve controller and a pump controller through a network. The method may increase a precision and/or the coordinate determination rate in response to an increase in elevation of the wastewater to account for increased volume per unit depth as the wastewater lagoon fills.

According to an embodiment, the method further includes generating a second geospatial coordinate at a second sensor buoy in a second wastewater lagoon wherein the second geospatial coordinate of the second wastewater lagoon comprises an elevation coordinate of the second geospatial coordinate, determining a wastewater depth of the second wastewater lagoon, determining that the second wastewater lagoon has a remaining capacity to accept discharge from the wastewater lagoon, and automatically initiating flow of wastewater from the wastewater lagoon to the second wastewater lagoon. The first coordinate determination rate may be at least as fast as one point per ten seconds.

### BRIEF DESCRIPTION OF THE DRAWINGS

The present invention will be more fully understood from the detailed description and from the accompanying drawings of various embodiments of the invention. The drawings should not be taken to limit the invention to the specific embodiments, but are for explanation and understanding only.

FIG. 1A illustrates a schematic diagram of a water level monitoring system including a sensor buoy deployed in a body of water and communicatively coupled through a network to a coordination server that monitors water levels, predicts capacity of the body of water, and generates alerts for a device of user and/or control instructions for infrastructure devices, according to one or more embodiments.

FIG. 1B illustrates a cross-sectional view of features of a prototypical body of water including water levels and capacity determinations, according to one or more embodiments.

FIG. 1C illustrates a top view of the body of water of FIG. 1B including site coordinates and perimeter contour data, according to one or more embodiments.

FIG. 2A illustrates a detailed view of a sensor buoy floating on water tethered to the floor of the body of water with an anchor, including a high-precision GPS unit able to determine a precise elevation coordinate, according to one or more embodiments.

FIG. 2B illustrates a block diagram of the sensor buoy components including geospatial positioning unit, a spatial correction unit, one or more other sensors, and wireless network interface controller, according to one or more embodiments.

FIG. 2C illustrates a detailed view of another instance of the sensor buoy 200, including use of cylindrical floats tied into a chassis, according to one or more embodiments.

FIG. 3 illustrates a system architecture diagram illustrating the coordination server, including a site profile storing one or more level functions usable to determine depth and/or volume based on elevation coordinates, according to one or more embodiments.

FIG. 4 illustrates a block diagram of device components of a device usable by the user such as a smartphone or tablet device, and which may include a monitoring application for receiving alerts and viewing current or predicted water levels, according to one or more embodiments.

FIG. 5 illustrates a cross-sectional view of irregularly shaped body of water with data quality requirements illustrated depending on a current depth of the body of water and which may be used to adjust accuracy, precision, time resolution of geospatial data, according to one or more embodiments.

FIG. 6 illustrates a hydraulic coupling system between multiple lagoons that is controllable through data gathered by one or more sensor buoys to automatically control the flow of water between lagoons, according to one or more embodiments.

FIG. 7 illustrates a working example embodiment in which a multi-lagoon wastewater treatment facility at a large-scale dairy including several communicatively coupled sensor buoys and/or control systems, according to one or more embodiments.

FIG. 8 illustrates a working example in which a sensor buoy in a treatment lagoon at a gas well remediation site is used to detect a leak in the treatment lagoon, according to one or more embodiments.

FIG. 9 illustrates a water body monitoring process flow, according to one or more embodiments.

FIG. 10 illustrates a flowchart of a site data collection process for geospatial coordinates from the sensor buoy, according to one or more embodiments.

FIG. 11 illustrates a flowchart for a site profile assembly process, according to one or more embodiments.

FIG. 12 illustrates a flowchart for a water monitoring alert process, according to one or more embodiments.

FIG. 13 illustrates a flowchart for an automatic precision and resolution adjustment process, according to one or more embodiments.

FIG. 14 illustrates a flowchart for setting up profiles usable for prediction including a usage profile, a climate profile, and/or a weather profile, according to one or more embodiments.

FIG. 15 illustrates a flowchart for a level prediction process, according to one or more embodiments.

FIG. 16 illustrates a flowchart for a hydraulic infrastructure control process, according to one or more embodiments.

Other features of the present embodiments will be apparent from the accompanying drawings and from the detailed description that follows.

### DETAILED DESCRIPTION

Disclosed are a method, a device, and/or system of water body monitoring and/or automatic control through a high-precision sensor buoy. Although the present embodiments have been described with reference to specific example embodiments, it will be evident that various modifications and changes may be made to these embodiments without departing from the broader spirit and scope of the various embodiments.

One or more of the present embodiments provide a comprehensive water level monitoring system that improves operational efficiency and/or environmental monitoring for wastewater lagoons, treatment facilities, cisterns, reservoirs, lakes, streams, estuaries, the ocean, and/or other bodies of water 100 through autonomous sensor buoys 200 equipped with high-precision geospatial positioning and optionally real-time communication capabilities. The systems, methods, and/or devices described herein may use one or more floating sensor buoys 200 that may be communicatively coupled to a centralized and/or off-site coordination servers 300 to deliver continuous, accurate, and/or precise water level measurements that enable predictive overflow prevention and/or automated infrastructure control.

One or more of the embodiments offer significant advantages over traditional monitoring approaches by providing an easily deployable, flexible, and/or mechanically simple solution. For example, in one or more embodiments, the sensor buoy 200 may use high-precision measurements using geospatial determination and correction (e.g., GPS in combination with RTK correction technology), and intelligent precision adjustment that may optimize energy efficiency based on operational conditions. In one or more embodiments, the sensor buoy 200 may operate easily in remote locations or otherwise away from existing power sources through use of solar power generation, a local correction base station, and/or a satellite internet backhaul (e.g., Starlink®, ViaSat®, Iridium®). In one or more embodiments, the system's ability to accurately calculate volumes in irregularly shaped bodies of water, combined with predictive analytics incorporating usage, weather and climate data, may help enable proactive management that prevents water waste and/or environmental incidents while supporting operational efficiency and/or regulatory compliance requirements.

Key advantages include easy deployment and provisioning, automated alert generation and infrastructure control capabilities that can prevent overflow events (and/or low water level alerts), coordinated management of multiple interconnected lagoons, real-time data transmission enabling immediate response to changing conditions, and/or comprehensive environmental monitoring through integrated sensors for water quality parameters. The system's scalable architecture can support deployment across multiple locations and/or sites while maintaining centralized oversight and data logging, making the system particularly valuable for large-scale operations such as wastewater treatment facilities (e.g., municipal sewage processing), agricultural operations (e.g., large scale dairies), and/or industrial water management systems (e.g., mines), for example that may require or benefit from reliable, autonomous monitoring with minimal maintenance requirements.

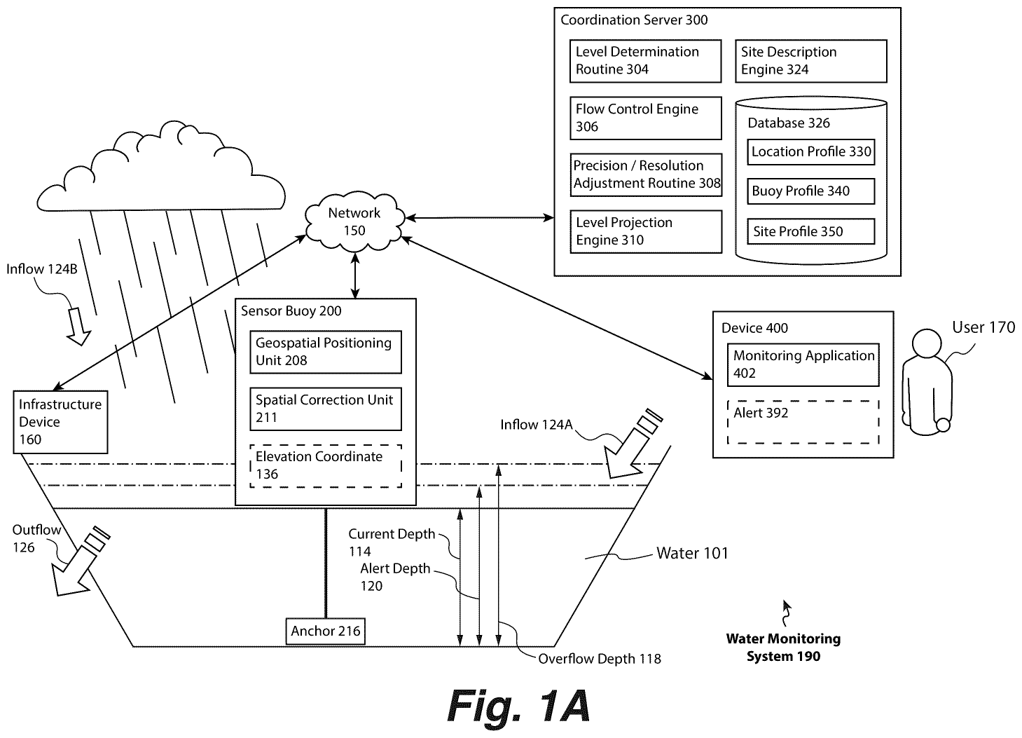

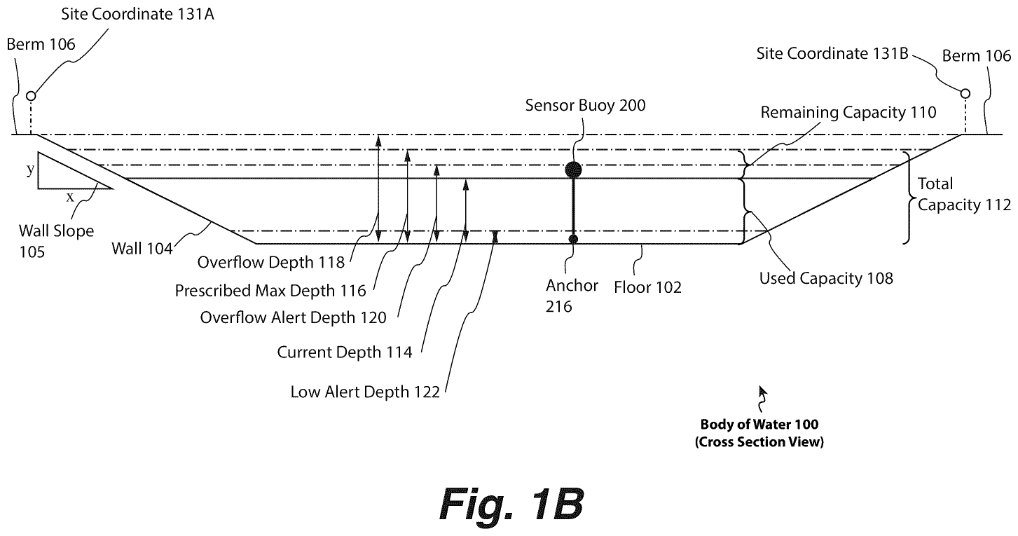

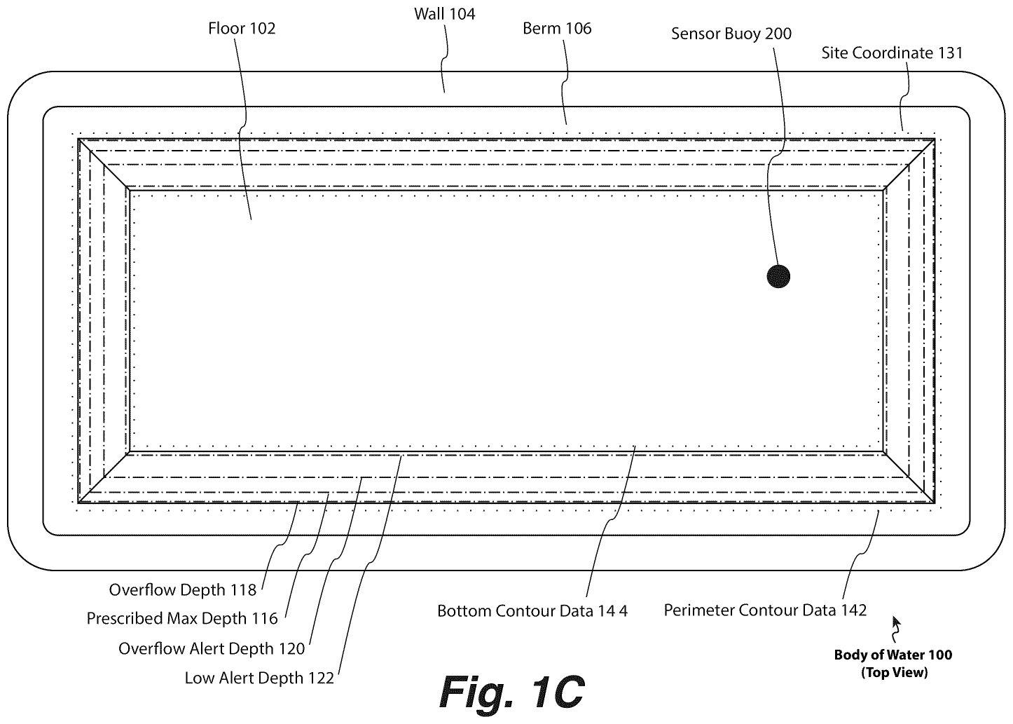

FIG. 1A illustrates a schematic diagram of a water monitoring system 190, according to one or more embodiments. In one or more embodiments, a sensor buoy 200 may be deployed in body of water 100. The body of water 100 may be, for example, a reservoir, lake, or wastewater lagoon. The sensor buoy 200 may be communicatively coupled through network 150 to coordination server 300. The network 150 for example may be implemented through one or more communication networks such as a cellular network (e.g., LTE, 5G), short range wireless protocols (e.g., WiFi), a digital radio network and protocol, and/or through a satellite connection (e.g., a low earth orbit satellite such as Starlink®). A user 170 interacts with the system through a device 400, such as a smartphone, tablet device, laptop, and/or desktop computer, which may include monitoring application 402. The coordination server 300 may communicate with infrastructure device 160 to provide automated control capabilities, as further shown and described throughout the present embodiments.

The sensor buoy 200 floats on the surface of body of water 100 at a current depth 114, and includes geospatial positioning unit 208, which may be based on Global Positioning System (GPS) or another mode of geospatial positioning. The geospatial positioning unit 208 may be mounted on elevation rod 204. The sensor buoy 200 may be free floating, or may be anchored to floor 102 via anchor 216 and/or direct fastening to an anchor point, allowing the sensor buoy 200 to maintain its position in a confined area while floating with water level changes.

Prior to deployment of the sensor buoy 200, initial site data may be collected by the sensor buoy 200 such that adequate data exists to determine the shape, size, and/or level functions (e.g., the level function 357) for the body of water 100, as further shown and described herein. For example, in one or more embodiments, the user 170 may use sensor buoy 200 as a survey rod (including possible detachment of the elevation rod 204) to gather perimeter, contour, and/or other geospatial data for the body of water 100 and its embracing site.

Coordination server 300 receives geospatial coordinate data from one or more instances of the sensor buoy 200 and processes this information using, for example, a level determination routine 304 to calculate water level, which may include depth and/or volume. The water level may include or may be used to determine used capacity 108 and/or remaining capacity 110. When threshold conditions are exceeded, the coordination server 300 may generate alerts 392 through a realtime alert system 309 and can automatically control one or more infrastructure devices 160 (e.g., a valve, a gate, a pump, a treatment system, etc.) through application of a flow control engine 306. This integrated approach may help enable continuous monitoring, including with predictive capabilities and automated response systems, which can improve operational efficiency, safety, and environmental compliance compared with traditional manual measurement methods by offering real-time data and early warning capabilities. The coordination server 300 can also improve operational efficiency and environmental compliance through automated infrastructure control for preventing overflow events.

The body of water 100 may experience both inflow 124 and outflow 126 which may change the current level of the body of water 100, for example current depth 114, current volume, and/or remaining capacity 110. In the present example, the inflow 124A may be from usage (e.g., adding wastewater to a wastewater lagoon) or may be through addition of other water sources which can find their way to the body of water 100, for example precipitation, groundwater percolation, or natural surface water runoff. As further shown and described throughout the present embodiments, usage may be evaluated to predict depth, volume, and/or capacity, including automatic initiation of alerts 392 and/or control instructions 396 responsive thereto.

The user 170 may be an individual tasked with monitoring and/or controlling a body of water 100, for example a farmer, an environmental regulator, a municipal wastewater worker, an industrial quality control specialist, an environmental scientist, a civil engineer, and/or other relevant personnel. The user 170 may be acting on behalf of themselves (e.g., as the owner of a farm), an organization such as a corporation or non-profit organization, and/or a government agency.

In one or more embodiments, the water monitoring system 190 described herein is applicable to various types of wastewater lagoons commonly used in agricultural, industrial, and municipal operations. These include anaerobic lagoons for primary treatment of high-strength organic waste, facultative lagoons that utilize both aerobic and anaerobic processes for secondary treatment, and aerobic lagoons for polishing effluent. The system may be particularly valuable for dairy lagoons that handle liquid manure and wash water, swine lagoons for managing hog waste, and/or poultry lagoons for processing chicken and turkey waste. Industrial applications include oil and gas wastewater lagoons for treating produced water and drilling fluids, mining lagoons for managing process water and tailings, evaporation ponds for collecting minerals (e.g., salt, lithium) and food processing lagoons for handling organic waste streams. Municipal applications encompass sewage lagoons for primary and secondary treatment, stormwater retention lagoons for managing urban runoff, and constructed wetlands for tertiary treatment. The system's precision geospatial monitoring and automated control capabilities may be especially beneficial for multi-stage lagoon systems where coordinated water level management across interconnected treatment cells is critical for maintaining optimal treatment efficiency and preventing environmental incidents.

It will be noted that in water level monitoring applications, precision and accuracy represent two distinct but important measurement characteristics that may directly impact the effectiveness of overflow prevention, infrastructure control, and/or environmental compliance systems. Accuracy refers to how close a measurement is to the true or actual water level value, while precision refers to the consistency and repeatability of measurements under identical conditions. For example, a sensor buoy 200 with high accuracy might consistently measure a water depth between 9.96 and 10.10 feet when the actual depth is 10.05 feet, whereas a sensor buoy with high precision would consistently produce the same measurement value (such as 9.80 feet) even if that value differs from the true depth. Good accuracy may help ensure that alerts 392 and/or control instructions 396 are triggered at the correct water levels, while sufficient precision may ensure that small but important changes in water level can be detected consistently over time, particularly important when monitoring shallow lagoons where small depth changes can represent significant volume changes. It should be noted that small changes in precision can result in relatively large inaccuracies in water volume due to multiplicative affect of depth, as further shown and described in conjunction with the embodiment of FIG. 5.

In one embodiments, the geospatial positioning unit 208 may be or include a GPS unit 209. GPS accuracy can be significantly enhanced through several complementary techniques, with coordinate averaging over time representing one of the most effective approaches for stationary or slowly-moving applications. By collecting multiple GPS readings over extended periods and calculating statistical averages, random errors caused by atmospheric interference, satellite geometry variations, and signal multipath effects often can be substantially reduced, which may improve accuracy from several meters to sub-meter precision. This temporal averaging technique may work particularly well when combined with Real-Time Kinematic (RTK) correction systems (e.g., an example of the spatial correction unit 211) that provide differential GPS corrections from nearby reference stations, enabling the sensor buoy 200 system to achieve centimeter-level accuracy essential for detecting small water level changes in shallow lagoons where minor depth variations can represent significant volume changes and potential overflow conditions. Both accuracy, precision, and/or data resolution (e.g., a number of elevation coordinates 134 per unit time) may be dynamically adjusted based on operational needs, as shown and described throughout the present embodiments.

FIG. 1B illustrates a cross-sectional view of a prototypical body of water 100, according to one or more embodiments. The figure demonstrates a sensor buoy 200 floating on the surface, which may enable precise determination of water depth and volume for regular (or irregularly shaped) bodies of water.

The cross-section of FIG. 1B shows floor 102 with wall 104 having wall slope 105, creating a regularly shaped containment structure typical of wastewater lagoons or treatment ponds. In one or more embodiments, the wall slope 105 may typically be a 2:1, 3:1, or 4:1 slope, which may depend on the intended wastewater, lagoon size, lagoon construction material, surrounding soil type, jurisdiction, and/or other factors. The sensor buoy 200 may be positioned on the water surface and optionally connected to an anchor 216 on floor 102 via a tether 214. The geospatial positioning unit 208 of the sensor buoy 200 (such as the GPS unit 209) may determine elevation coordinate(s) 136. The elevation coordinate 136 may be transmitted to and/or processed by coordination server 300 to calculate current depth 114, which may represent a vertical distance from the water surface to floor 102.

FIG. 1B also illustrates division of the body of water 100 into several operational zones for monitoring and alert purposes. Used capacity 108 represents the current volume of water 101 in the body of water 100, while remaining capacity 110 shows available storage before reaching the prescribed maximum depth 116. Critical monitoring thresholds include low alert depth 122 (e.g., alerting at a minimum operational level), overflow alert depth 120 for early warning of potential overflow conditions, and/or prescribed maximum depth 116 representing the maximum safe or regulatorily prescribed operating level. The overflow depth 118 indicates the point at which water would breach or overlap the reservoir, lagoon, dam, and/or other containment structure. Although one overflow alert depth 120 is shown, it will be understood that multiple instances of the overflow alert depth 120 may be specified, and/or alerts or pushed updates specified at certain capacities (e.g., each foot of depth which the body of water 100 rises or falls, each acre-foot of water volume which enters or leaves body of water 100, each change in 10% capacity, etc.).

The cross-sectional view of FIG. 1B also demonstrates how precise elevation measurements may enable accurate volume calculations even in irregularly shaped bodies of water where traditional depth measurements may be insufficient. For example, reference is made to the irregular shape of FIG. 5, the contour of which may result from draws or canyons flooded by a dam. Such natural contours of a body of water 100 may cause an irregular and/or non-linear volume-per-unit-depth function. In one or more embodiments, the coordination server 300 may use the elevation data (e.g., one or more instances of the elevation coordinate 136) combined with estimated, pre-surveyed, or post-deployment surveyed contour information of the floor 102 of the body of water 100 to determine the current depth 114 and/or the corresponding volume, remaining capacity 110, geographical water extents, and/or proximity to critical thresholds such as the prescribed maximum depth 116. This capability may provide significant advantages over conventional monitoring systems by enabling predictive overflow prevention and automated capacity management for environmental compliance and/or operational efficiency.

In one or more embodiments, the body of water 100 may be entirely defined from existing data, for example existing LIDAR contours (e.g., routinely shot by the Army Corps of Engineers from helicopters over wide areas), defined in geospatial databases, through reference to engineered permit plans, or other available sources. For example, a user 170 may manually bound the body of water 100 on a geospatial viewing and/or processing application (e.g., offered by Esri®, Google Earth, etc.).

In one or more other embodiments, the body of water 100 and contours thereof may be entirely collected by the users 170. For example, the user 170 may use the sensor buoy 200 and/or detachable elevation rod 204 to traverse the empty or substantially empty site to collect site coordinates 131 including contour data on the floor 102, and perimeter data of the extent of the body of water 100 (e.g., the perimeter contour data 142). A resulting approximate dimensional representation of the body of water 100 and its possible extents may then be generated. This may represent an advantage by sing a single piece of equipment to quickly and easily set up a new site for a body of water 100.

In one or more embodiments, a simple combination of gathered and existing data may be used to streamline defining the body of water 100, which may be especially efficient for permitted, engineered, and/or relatively regularly shaped bodies of water 100. For example, in one or more embodiments, the user 170 and/or a robotic or drone vehicle may gather perimeter contour data 142 along a berm 106, which may include elevation data. Engineering specifications may then be queried and/or entered defining the wall slope 105 and/or wall length or total depth of the body of water 100, which together may be used to generate a complete three dimensional description, contour, model, and/or mathematical representation of the body of water 100 and its possible water levels.

FIG. 1C illustrates a top view of the body of water 100, for example the body of water 100 of FIG. 1B, showing the site coordinates 131, perimeter contour data 142, and bottom contour data 144, according to one or more embodiments. FIG. 1C demonstrates the comprehensive spatial mapping capabilities that enable the water level monitoring system to accurately calculate volumes and depths for both regularly and irregularly shaped bodies of water 100 through precise geospatial coordinate collection and contour analysis. Additional aspects such as the prescribed maximum depth 116 may also be determinable from permit, engineering data, and/or other sources.

In one or more other embodiments, and as further shown in FIG. 1C, a complete description of the body of water 100 may be generated by gathering both the bottom contour data 144 and the perimeter contour data 142, where the wall slope 105 can be determined through calculation of an approximate slope connecting points, lines, or polygons generated for each of the bottom contour data 144 and the perimeter contour data 142.

The top view of FIG. 1C shows the complete perimeter outline of a body of water 100, which may be gathered with a sensor buoy 200 or portion thereof, when operating in a site description capacity. FIG. 1C also displays site coordinate 131 markers that may define the precise boundaries and shape characteristics of the body of water 100. Perimeter contour data 142 provides detailed boundary information, while bottom contour data 144 may represent the floor 102 topology.

The spatial mapping shown in FIG. 1C demonstrates how the system can collect comprehensive site data during the initial deployment, provisioning, and/or configuration phases. The perimeter contour data 142 may define the exact boundaries of the body of water 100, either at the prescribed maximum depth 116, the overflow depth 118, or some other level, which may enable the coordination server 300 to establish precise level functions 357. The bottom contour data 144 may provide the three-dimensional floor profile necessary for calculating water volumes at different depths, particularly important for natural bodies of water with irregular bottom topography. Although the bottom contour data 144 is shown as a perimeter and/or polygon in FIG. 1C, a complete contour of the floor 102 may be collected and used for an irregular or sloped floor 102, even walls 104 and/or berm 106 holding the body of water 100 are relatively regular or geometrically shaped. For example, it may be common for lagoons to be sloped on their bottom, irregularly build up sediment, or have other unique ground irregularities.

The site coordinate 131 points may include a geospatial coordinate 130 defining the site in and around the body of water 100. The site coordinates 131 may work in conjunction with the geospatial positioning capability of the sensor buoy 200 to provide accurate elevation data for the water level of the body of water 100. The comprehensive mapping enables the system to generate precise depth function 358 and/or volume function 359, which may account for the irregular shape and/or varying depth characteristics of the body of water 100.

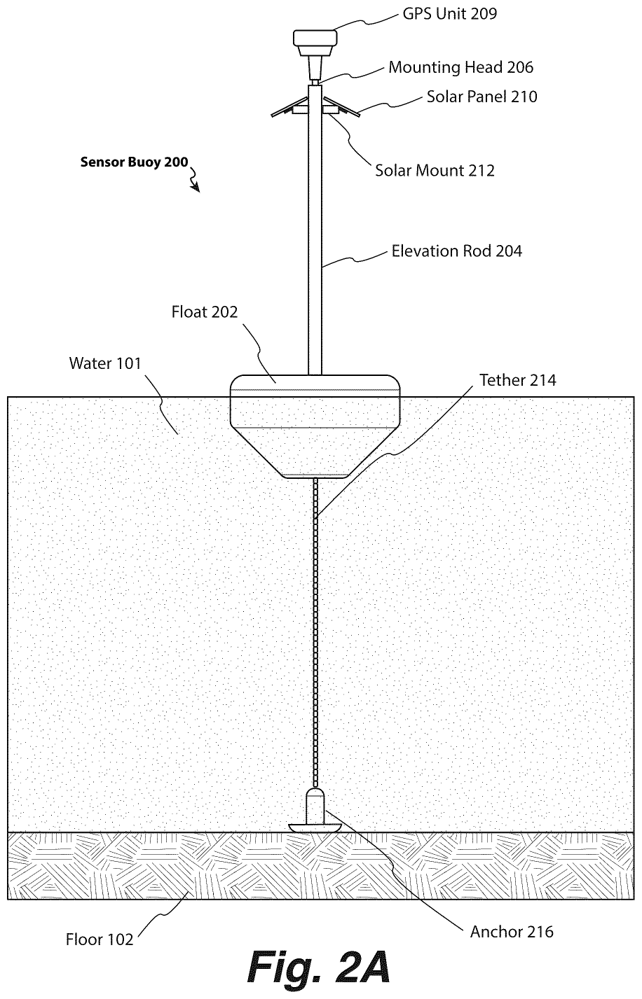

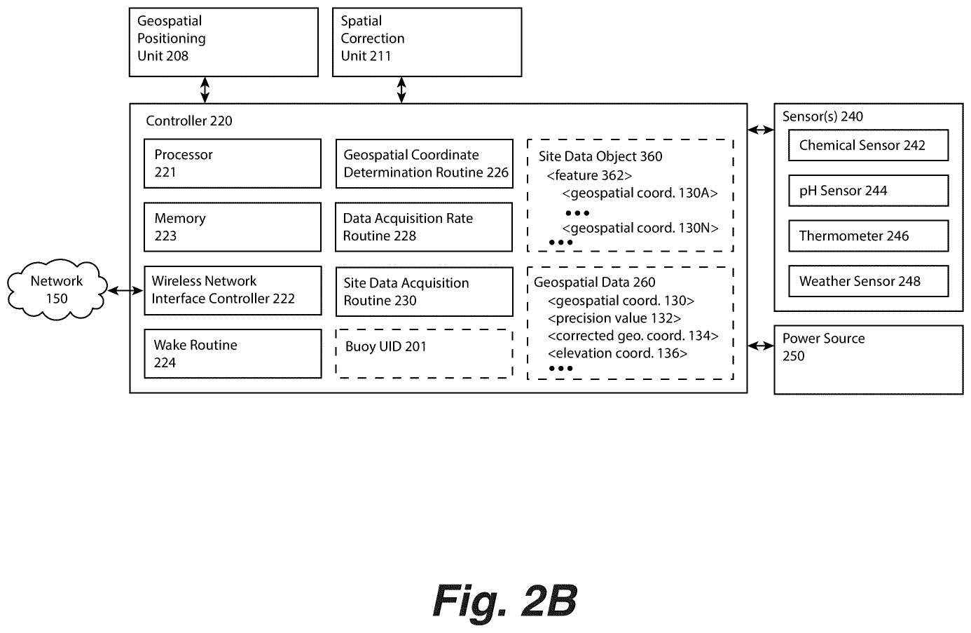

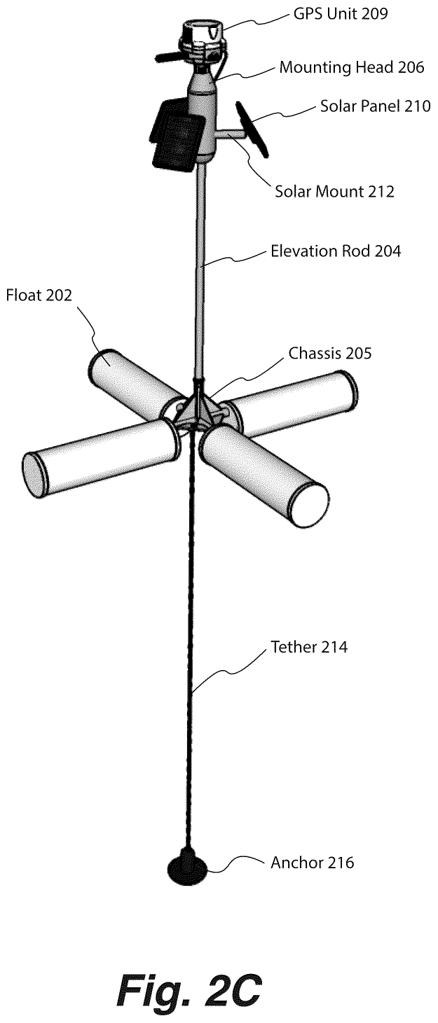

FIG. 2A illustrates a detailed view of one possible instantiation of the sensor buoy 200 floating on water 101 of the body of water 100, according to one or more embodiments. FIG. 2A shows an example of a physical construction and deployment configuration of the sensor buoy 200, including demonstrating how the various components are integrated to help provide stable water level monitoring capabilities.

The sensor buoy 200 comprises a float 202 that provides buoyancy to maintain the sensor buoy 200 on the water surface of the body of water 100. The float 202 may be made of any suitably buoyant materials such as styrofoam, a waterproof plastic or metal enclosure filled with air, a rigid shell of plastic or metal filled with closed-cell foam, and/or other floats or sources of buoyancy known in the art. In one or more embodiments, the float 202 may be selected to be of appropriate width and/or weight to resist tipping in wind or waves and/or ballast the sensor buoy 200.

In one or more embodiments, the elevation rode 204 may provide a fixed spatial relation to the surface of the water from the geospatial positioning unit 208. Although the elevation rod 204 and/or buoyancy of the float 202 may be calibrated for fresh water, adjustments may be made to accommodate certain liquid densities such as highly saturated salt water, to ensure accuracy of elevation coordinates 136.

Elevation rod 204 may be coupled to float 202 and may extend vertically upward, which may help provide a stable mounting platform for precision measurement equipment and/or improved communication network reception. Improved communication network reception may be helpful, for example in flat locations where a cell tower connected to the wireless network interface controller 222 may be distal to sensor buoy 200. For example, the elevation rod 204 may help alleviate any network interference from the berm 106 as the current depth 114 falls. At the top of elevation rod 204, a mounting head 206 may secure a geospatial positioning unit 208 (e.g., the GPS unit 209) in an optimal position for satellite signal reception (e.g., GPS and/or GLONASS satellites). The sensor buoy 200 may include a power source 250 such as the solar panel 210, which can be mounted on solar mount 212, which may be attached to the elevation rod 204 to provide renewable power generation and/or charging a battery. The elevation rode 204 can be of arbitrary length provided sufficient anchoring, buoyancy of the float 204, and/or ballast are provided.

An anchoring system may help ensure the sensor buoy 200 maintains an intended position while allowing it to float freely along a z-axis (e.g., elevation) with changing water levels. In one or more embodiments, the sensor buoy 200 may be anchored to a deepest portion of the body of water 100 to maximize the available information as to depth. A tether 214 may connect the float 202 to an anchor 216, which is placed and/or secured to floor 102 of the body of water 100. In one or more other embodiments, the sensor buoy 200 may be constrained in its movement through other methods, for example confinement in a cage structure, attachment to other buoys or floating structures (e.g., a floating dock), and/or other methods which will be evident to one skilled in the art. This configuration allows the sensor buoy 200 to rise and fall with water level changes while preventing the sensor buoy 200 from drifting away from its designated monitoring location. A length of the tether 214 may comprise rope, cable, chain, and/or another suitable material. In one or more embodiments, the float 202, the tether 214 and/or the anchor 216 may be selected to resist any corrosive potential of the body of water 100, such as acids, bases, minerals, bacteria, algae, barnacles, mollusks, or and/or other hazards or nuisances. For example, in one or more embodiments the tether 214 may be made from plastic, PFAS polymers (e.g., for extreme environments), and/or stainless steel.

Although not shown, the controller 220 of FIG. 2B may be housed in a number of locations, including at the top of the elevation rod 204, within the elevation rod 204, within the float 202, and/or in another suitable location. Additional sensors 240 may be included in a relevant location to the purpose of the sensor 240. For example, a pH sensor 244 may be positioned on the float 202 such that the pH sensor 244 is in contact with the water 101 for continual or periodic acidity and/or basicity sensing. Other sensors can include detectors and/or measurers of specific chemicals, heavy metals, volatile compounds, nitrogen or phosphorus compounds, hydrocarbons, total dissolved solids, turbidity, water clarity, absorption spectra, and/or other chemical constituents or environmental contaminants. In one or more embodiments, the sensors 240 may include a water quality sensor such as an oxygenation sensor (e.g., dissolved O2), a nitrate sensor, a phosphate sensor, a pathogen sensor (e.g., E. coli), and/or a heavy metal or metalloid sensor (e.g., lead, arsenic, cadmium, lead, and mercury, etc.).

FIG. 2B illustrates a block diagram of the sensor buoy 200 components including the controller 220 and included and/or peripheral communicatively coupled elements, according to one or more embodiments. FIG. 2B illustrates internal electronic components of the sensor buoy 200, including demonstrating how the various subsystems may work together to provide autonomous water level monitoring capabilities and other features and functions described herein, according to one or more embodiments. In one or more embodiments, the sensor buoy 200 may provide significant advantages over traditional monitoring systems by combining easy deployment, flexible configuration, precise positioning, comprehensive sensing, autonomous operation, and/or real-time data transmission in a single deployable unit powered by the controller 220.

The sensor buoy 200 may include several key electronic components that may be integrated into a cohesive monitoring system. Controller 220 may include a processor 221 (e.g., a microcontroller, a CPU, a PCB with one or more processing modules, etc.) for executing monitoring algorithms. Controller 220 may also include a wireless network interface controller 222 for communication with coordination server 300 via a wireless network (e.g., the network 150 or portion thereof). For example, the wireless network interface controller 222 may communicate through cellular protocols (e.g., 3G, 4G, LTE, 5G), digital radio, WiFi, Bluetooth, and/or other wireless protocols to access the network 150. The network 150 may be one or more networks, including without limitation a cellular network, a virtual private network (VPN), a local area network (LAN), a wide area network (WAN), and/or the internet. Memory 223 stores operational software including for example: a wake routine 224 for power management, a data acquisition rate routine 228 for optimizing measurement frequency based on operational requirements, a geospatial coordinate determination routine 226 for determining geospatial data including under operational conditions, a site data acquisition routine 230, and/or a buoy UID 201 for unique identification. The memory 223 may also store one or more site data objects 360, and/or one or more sets of gcospatial data 260.

The controller 220 may include and/or may be communicatively coupled with the geospatial positioning unit 208 (for example, GPS unit 209) for satellite-based or non-satellite based location determination. In one or more embodiments, a non-GPS system may be used, including determination with respect to a local position with a known coordinate. For example, in one or more embodiments, the relative location to a fixed reference point (e.g., located on the berm 106) may be used. The positioning unit 208 may be enhanced by a spatial correction unit 211 that may process baseline geospatial data to generate precise, e.g., corrected, geospatial coordinates (e.g., the corrected geospatial coordinate 134), including elevation coordinates 136. In one or more embodiments, the spatial correction unit 211 may include an RTK system that may receive correction data from an outside source, such as via a cellular network.

Real-Time Kinematic (RTK) correction may represent an enhancement to standard GPS positioning that may improve accuracy from several meters to centimeter-level precision. RTK correction works by utilizing a network of fixed reference stations with precisely known coordinates that continuously receive GPS satellite signals and calculate the difference between their actual position and the GPS-calculated position. These correction signals, which can account for atmospheric delays, satellite orbit errors, and other sources of positioning uncertainty, may be transmitted in real-time to mobile GPS receivers (e.g., via the network 150) such as the GPS unit 209. The spatial correction unit 211 may process these correction signals to generate highly accurate geospatial coordinates (e.g., the corrected geospatial coordinate 134) usable for precise location measurements, enabling the system to detect even small changes in water elevation that would be difficult or unreliable with standard GPS accuracy. For example, especially in relatively large and/or broad shallow bodies of water 100, the standard error of GPS may cause highly inaccurate readings that might prevent detection of an overflow or low level event, cause false positives for alerts, and/or provide inaccurate usage over time data.

The dual-component approach using GPS and RTK may therefore provide high-accuracy and high precision positioning measurements that may help enable accurate and/or precise water level calculations in the body of water 100.

Additional sensor capabilities are provided through sensor 240, which can include various environmental monitoring devices such as chemical sensor 242 for water quality assessment, pH sensor 244 for acidity monitoring, thermometer 246 for temperature measurement, and weather sensor 248 for atmospheric conditions such as windspeed, humidity, and/or solar intensity. These sensors enable comprehensive environmental monitoring beyond basic water level measurement. It should be noted that sensed values and/or thresholds thereof may be used in combination for alerts 392 and control instructions 396. As just one example, overflow of body of water 100 may not matter if a chemical is below a threshold value such that the overflow would not be environmentally threatening. In another example, automatic pumping from one wastewater lagoon with a high concentration of a chemical to a second wastewater lagoon with a low concentration of the chemical will only be permitted to the extent that the concentration of the second wastewater lagoon would be calculated to remain below a certain threshold concentration.

Power management may be handled by power source 250. The power source 250 can include a variety of sources, for example a battery and/or a wired power source. In one or more embodiments, the power source 250 may be both a battery and/or the solar panel system shown in FIG. 2A. Other power sources 250 may include wind power, or even chemical and/or electrolytic potential of the body of water 100 depending on which chemicals are present. The power source 250 may work in conjunction with the wake routine 224 to optimize energy consumption by cycling between active monitoring periods and low-power standby modes, ensuring long-term autonomous operation with minimal or no manual intervention.

The controller 220 may generate and/or store site data object 360 for storing collected measurements and/or site information. The system controller 220 processes geospatial coordinates 130, sensor readings, and operational parameters into structured data formats that can be transmitted to coordination server 300 for analysis, alert 392 generation, and/or control instruction 396 generation.

In one or more embodiments, the controller 220 may collect and store a site data object 360 which may describe one or more features 362 of the site of the body of water 100. In one or more embodiments, the site data object 360 may merely include a collection of geospatial coordinates 130 (which may include elevation coordinates 136) which describe a surface topology of an area that contains or will contain the body of water 100. In one or more other embodiments, specific features 362 may be defined, for example perimeters (e.g., the perimeter contour data 142), the toc of a slope (e.g., the bottom contour data 144 around the interior edge of the wall 104), the crest of a berm 106, etc. The site data object 360 and/or features 362 or geospatial coordinates 130 thereof may be collected at an increased data collection rate (e.g., one coordinate per second, one coordinate per five seconds, etc.) relative to normal operation of the sensor buoy 200 in order to collect data as the user 170 moves to collect the site data object 360 (e.g., walks or uses a vehicle to convey the geospatial positioning unit 208).

Once the sensor buoy 200 is placed and enters an “operational mode” for water level sensing, the controller 220 may generate and store multiple instances of geospatial data 260 each representing a collection point and/or a data snapshot. The geospatial data 260 may be periodically collected and processed on the controller 220 and/or at the coordination server 300. For example, in one or more embodiments, several instances of the geospatial data 260 may be collected and averaged before sending over the network 150. The geospatial data 260 may include a geospatial coordinate 130, a precision value 132, a corrected geospatial coordinate 134 (which may have been adjusted with correction data), and/or an elevation coordinate 136. In one or more embodiments, the corrected geospatial coordinate 134 and/or any elevation coordinate 136 thereof may be accurate to around ±1 cm or ±2 cm depending on the quality of the correction data.

In one or more embodiments, the format of the geospatial coordinates 130 may include a NMEA (National Marine Electronics Association) string format. NMEA strings represent a standardized data format used by GPS receivers and other marine electronic devices to communicate positioning, navigation, and timing information. The NMEA format provides structured ASCII sentences that contain specific data fields including latitude, longitude, elevation, time stamps, satellite information, and positioning quality indicators. For GPS applications, common NMEA sentence types include GGA (Global Positioning System Fix Data) which provides position coordinates and fix quality, and RMC (Recommended Minimum) which includes position, velocity, and time data. The NMEA format enables standardized communication between the geospatial positioning unit 208 and the controller 220, facilitating consistent data parsing and processing regardless of the specific GPS hardware manufacturer. This standardization may be particularly valuable for the sensor buoy 200 system for compatibility with various GPS units and RTK correction systems while providing the structured data format necessary for precise water level calculations and coordination server 300 communication.

The wake routine 224 represents a power management system that enables the sensor buoy 200 to operate autonomously for extended periods while maintaining precise water level monitoring capabilities related to operational needs, including in conjunction with the data acquisition rate routine 228, as further shown and described herein. The wake routine 224 may implement a sleep-wake cycle that balances consistent monitoring requirements with energy conservation needs, particularly important for solar-powered deployments in remote locations where battery life directly impacts operational reliability. The wake routine 224 may operate by receiving instructions to set configurable timer intervals during which the sensor buoy 200 enters a low-power standby mode, reducing energy consumption by powering down non-essential systems while maintaining core functionality and/or an ability to wake upon expiration of the timer. During the low-power mode, the controller 220 minimizes power draw from the battery which may extend autonomous operation time significantly compared to continuous active monitoring. When the timer expires, the wake routine 224 automatically transitions the sensor buoy 200 to active mode, powering up the geospatial positioning unit 208, wireless network interface controller 222, and other sensors 240 to collect and transmit water level data.

In one or more embodiments, the wake routine 224 may include computer readable instructions that when executed: set a timer (e.g., within a microcontroller or other timing circuit); initiate a low power mode; determine expiration of the timer; initiate an active mode; and determine the first geospatial coordinate 130 and the first precision value 132 from the geospatial positioning unit 208 upon entering the active mode to increase energy efficiency of the power source 250.

The geospatial coordinate determination routine 226 may be configured to receive geospatial data 260 and/or a geospatial coordinate 130 from the geospatial positioning unit 208, along with any correction data from the spatial correction unit 211, and may generate the corrected geospatial coordinate 134 (which may include the elevation coordinate 136).

In one or more embodiments, the geospatial coordinate determination routine 226 may include computer readable instructions that when executed: determine a first geospatial coordinate 130 and a first precision value 132 from the geospatial positioning unit 208; receive correction data from the spatial correction unit 211; generate a corrected geospatial coordinate 134; and transmit the corrected geospatial coordinate 134 (which may include the elevation coordinate 136) to the server (e.g., the coordination server 300) over the wireless network 150.

The data acquisition rate routine 228 represents a power management and measurement optimization routine that enables the sensor buoy 200 to dynamically adjust its data collection frequency based on operational conditions, data resolution requirements, data accuracy requirements, and/or data precision requirements. This routine may work in conjunction with the wake routine 224 to balance measurement accuracy with energy efficiency, ensuring long-term autonomous operation while maintaining the monitoring precision necessary for accurate water level determination and overflow prevention.

According to one or more embodiments, the data acquisition rate routine 228 may operate by continuously evaluating current water level conditions, volume per unit depth characteristics (e.g., as shown in conjunction with the embodiment of FIG. 5), and/or proximity to critical thresholds (e.g., the prescribed maximum depth 116) to determine optimal data collection rate. During periods of stable water levels and/or when the body of water 100 is at low depths where volume changes per unit depth may be minimal (depending on the contour of the body of water 100), the data acquisition rate routine 228 may reduce the frequency of geospatial coordinate 130 collection to conserve the power source 250 such as a battery. For example, accuracy may be decreased by averaging fewer collected geospatial coordinates 130, correction may be turned off, and/or time-resolution may be decreases so that fewer points need to be determined. Conversely, when water levels approach critical thresholds or when volume changes more rapidly per unit depth (such as when a shallow lagoon approaches full capacity), the data acquisition rate routine 228 may automatically increase data acquisition rates to ensure adequate precision for detecting rapid changes and/or generating timely alerts 392 or control instructions 396.

The routine may also coordinate with the coordination server 300 to receive data quality requirement updates based on real-time analysis of water level data and operational conditions. When the coordination server 300 determines that current data quality requirements are not being met due to changing conditions, the coordination server 300 can transmit instructions to increase the data acquisition rate, coordinate resolution, and/or the quantity of geospatial coordinates 130 averaged to determine final positioning data. Such a server-coordinated approach may assist in maintaining optimal measurement precision across varying operational conditions, outsourcing power-intensive processing functions to the coordination server 300 while supporting centralized management of multiple deployed sensor buoys 200.

The data acquisition rate routine 228 may provide significant advantages for autonomous water level monitoring by enabling intelligent adaptation to changing operational requirements with little or no manual intervention. In one or more embodiments, the data acquisition rate routine 228 may include computer readable instructions that when executed: receive a reduced data quality requirement request (e.g., from the coordination server 300) in response to a drop in a depth of the body of water 100, and configure a coordinate determination rate that (i) slows the rate at which geospatial coordinates 130 are determined to increase energy efficiency of the power source 250 and/or (ii) reduces a quantity of geospatial coordinates 130 gathered for calculating average geospatial coordinates 130.

In one or more embodiments, the site data acquisition routine 230 may be configured to enable the sensor buoy 200 to function as a comprehensive site surveying tool for gathering detailed topographical and/or boundary information about the body of water 100 prior to deployment for water level monitoring. This routine may operate by receiving a request to initiate a site acquisition mode, either from the user 170 on site (e.g., via a site survey button on the sensor buoy 200 or the device 400 with a companion software App) and/or an instruction received from the coordination server 300, then configuring either a continuous point acquisition mode that determines geospatial coordinates 130 at a relatively rapid rate (e.g., at least one point per ten seconds), or a manual point acquisition mode for precise boundary marking. For example, in some embodiments, just the four corners of a rectangle lagoon may be marked. The routine may initiate a site data object 360 for the body of water 100 and systematically gathers site data comprising a first set of geospatial coordinates 130 (including any elevation coordinate 136), with each geospatial coordinate 130 optionally paired with a precision value 132 and any correction data to help ensure data quality and/or quality control. Upon receiving a request to end the site acquisition mode, the site data acquisition routine 230 may commit the collected site data to the site data object 360 and/or transmit the site data object 360 or portion thereof to the coordination server 300 over the wireless network 150. This capability may be used to transform the sensor buoy 200 into a dual-purpose device that can both survey the water body's characteristics during initial setup and subsequently monitor water levels during operational deployment, eliminating the need for separate surveying equipment and ensuring that the same high-precision geospatial positioning system used for water level monitoring also can be used for site characterization.

In one or more embodiments, the site data acquisition routine 230 may include computer readable instructions that when executed: receive a request to initiate a site acquisition mode to gather site data for the body of water 100; configure (i) a continuous point acquisition mode determining geospatial coordinates 130 at a first coordinate determination rate at least as fast as one point per ten seconds and/or (ii) a manual point acquisition mode; initiate a site data object 360 for the body of water 100; gather the site data comprising a first set of geospatial coordinates (each geospatial coordinate 130 of the first set of geospatial coordinates 130 paired with a precision value 132); receive a request to end the site acquisition mode gathering the site data for the body of water 100; commit the site data to the site data object 360; and/or transmit the site data object 360 to the server (e.g., the coordination server 300 or another server) over the wireless network 150.

FIG. 2C illustrates another instantiation of the sensor buoy 200, according to one or more embodiments. Four cylindrical floats 202 may be radially distributed and coupled to the elevation rod 204 through a chassis 205 which may be made of plastic, metal, carbon fiber, or another suitable material. The radial distribution of the floats 202 may help prevent tipping or wobbling in the body of water 100, further increasing accuracy and precision during geospatial data generation. The solar mount 212 may include three mounting locations for three solar panels 210, including radial distribution such that at least one solar panel 210 is likely to have an advantageous angle relative to the sun for adequate power generation. The battery may be housed in the mounting head 206, the GPS unit 209, and/or within the floats 202, according to one or more embodiments.

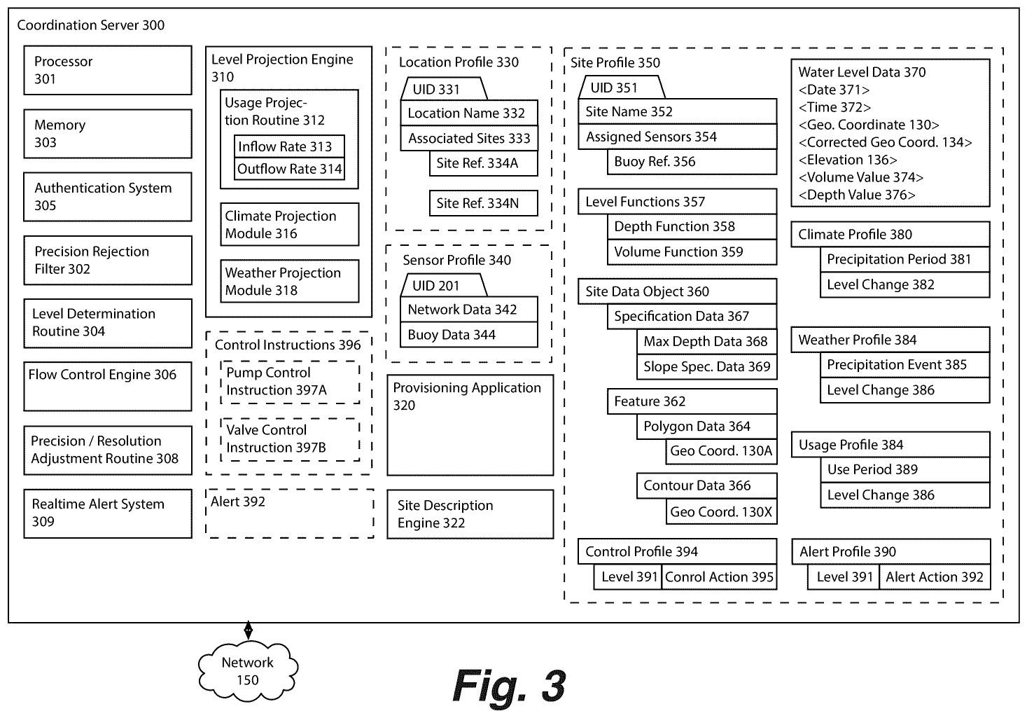

FIG. 3 illustrates a system block diagram showing the coordination server 300, according to one or more embodiments. In one or more embodiments, FIG. 3 demonstrates collection of elements that may enable a centralized water level monitoring and control across multiple sensor buoys 200 and connected infrastructure devices 160. Although shown as a single server, it will be appreciated the coordination server 300 may be implemented as one or more physical or virtual servers, which may be stored in one or more physical locations or data centers.

The coordination server 300 may serve as a central hub of the monitoring system, and may include processor 301 for executing system operations and memory 303 that is a non-transitory computer readable medium for storing operational software and data. The coordination server 300 may include several specialized processing engines, routines, subroutines, and/or modules that may work together to provide comprehensive sensor buoy 200 management, water level monitoring, water level prediction, and/or water infrastructure control capabilities.

In one or more embodiments, the coordination server 300 may include a precision rejection filter 302 for validating measurement accuracy.

The precision rejection filter 302 may be used for data quality control to filter geospatial data such that geospatial coordinates 130 meet specified accuracy and/or precision requirements for water level calculations, control instruction 396 generation, and/or alert 392 generation. In one or more embodiments, precision rejection filter 302 may operate by evaluating the precision values 132 associated with each geospatial coordinate 130 and/or corrected geospatial coordinate 134 received from the sensor buoy 200, and comparing such one or more geospatial coordinates 130 against predetermined precision requirements (e.g., that may vary based on current water conditions and operational needs). When a geospatial coordinate 130 fails to meet the required precision threshold, the precision rejection filter 302 may automatically discard (and/or store but discount for use) the measurement to prevent inaccurate water level determinations that could result in false alerts or missed overflow conditions. As just one example, the precision requirement may be 5 cm or less if a current depth 114 of a wastewater lagoon is one meter or more away from a prescribed maximum depth 116, and 2 cm or less if within one meter of the prescribed maximum depth 116. The precision rejection filter 302 may include logic to track the frequency of rejected measurements within specified time periods, and when rejection rates exceed acceptable limits, the precision rejection filter 302 generate a procedure call to the data quality adjustment routine 308, automatically instruct the sensor buoy 200 to increase its coordinate determination rate (e.g., coordinate resolution) and/or the quantity of measurements averaged to improve data quality. As a result, the precision rejection filter 302 water level monitoring system may help maintain high precision and/or accuracy while providing feedback mechanisms to optimize sensor performance, which may be particularly important for applications requiring precise measurements such as overflow prevention in wastewater lagoons and environmental compliance monitoring.

In one or more embodiments, the precision rejection filter 302 includes computer readable instructions that when executed: read the precision value 132 upon receipt of the corrected geospatial coordinate 134 (and/or the geospatial coordinate 130); determine the precision value 132 does not meet a data quality requirement (e.g., a data quality requirement 500, for example as shown in FIG. 5); optionally delete the corrected geospatial coordinate 134 and/or flag the corrected geospatial coordinate 134 as inaccurate; and/or optionally increase a coordinate determination rate of the sensor buoy 200 (e.g., through communication with the data acquisition rate routine 228).

In one or more embodiments, the coordination server 300 may include a level determination routine 304 for determining a level of the water 101 in the body of water 100, for example either a depth or volume. In one or more embodiments, the level determination routine 304 may be configured to transform raw geospatial coordinate 130 data from sensor buoys 200 (e.g., from the geospatial data 260) into actionable water level information including depth and/or volume calculations.

In one or more embodiments, the level determination routine 304 may operate by receiving geospatial coordinates 130 and/or elevation data 136 (either or both of which may have been corrected or subject to correction data) from deployed sensor buoys 200, querying site profiles 350 containing level functions 357 (such as depth functions 358 and/or volume functions 359) specific to each body of water 100 which may account for the particular dimensions or unique topographical characteristics of lagoons, reservoirs, and/or treatment facilities. The level determination routine 304 may input elevation coordinates 136 into a depth function 358 to calculate current water depth (e.g., the current depth 114). Alternatively, or in addition, the level determination routine 304 may input elevation coordinates 136 into a volume function 359 to calculate current volume and/or used capacity 108.

In one or more embodiments, the level determination routine 304 may include computer readable instructions that when executed: receive the first geospatial coordinate 130 and the correction data and/or receive the corrected geospatial coordinate 134 from the sensor buoy 200; query a site profile 350 of the body of water 100; and determine a level of the body of water 100. Determining the level of the body of water 100 may include (i) inputting the elevation coordinate into a depth function 358 for a wastewater lagoon and determining a depth of wastewater (e.g., the water 101) in the wastewater lagoon, and/or (ii) inputting the depth of the wastewater into a volume function 359 of the wastewater lagoon generated based on a contour map (e.g., the contour data 366) of the wastewater lagoon and determining a volume of the wastewater (e.g., the water 101) in the wastewater lagoon.

The authentication system 305 may be configured to authenticate any of the devices, servers, and/or other communicating elements of the water monitoring system 190. For example, each of the sensor buoys 200 may be registered and identified by unique identifier (e.g., the buoy UID 201), authenticated with stored credentials, and/or verified using digital certificates or other means known in the art of cybersecurity. In one or more embodiments, the authentication system 305 may help ensure only authorized sensor buoys 200 can communicate with the coordination server 300 and transmit water level data to the coordination server 300. This authentication system may operate by verifying the identity of each sensor buoy 200 through unique identifiers, digital certificates, or cryptographic keys before allowing data transmission of geospatial coordinates 130 and/or accepting control instructions 396. The authentication system 305 may prevent unauthorized devices from accessing the water level monitoring system 190, protect against data tampering or false readings that could compromise overflow prevention systems, and/or ensure the integrity of critical infrastructure control commands sent to valves, pumps, and other automated equipment. This security capability is particularly relevant to wastewater treatment facilities, industrial operations, energy facilities (e.g., dams with hydroelectric generation potential) and municipal water management systems where unauthorized access could result in environmental incidents, operational disruptions, infrastructure outages, facility damage, and/or regulatory compliance violations.

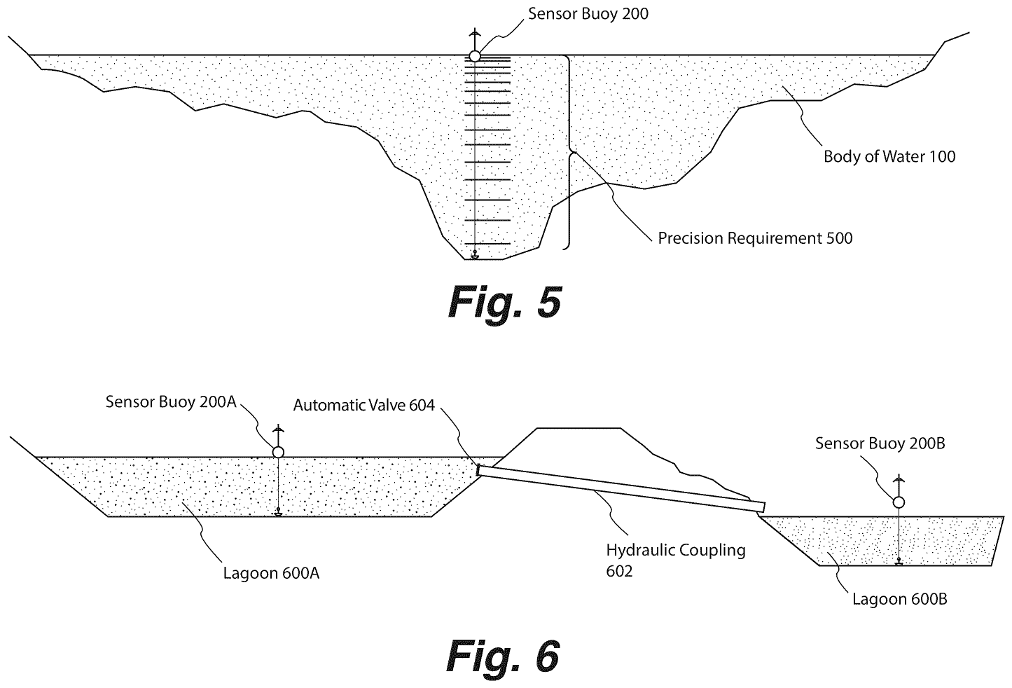

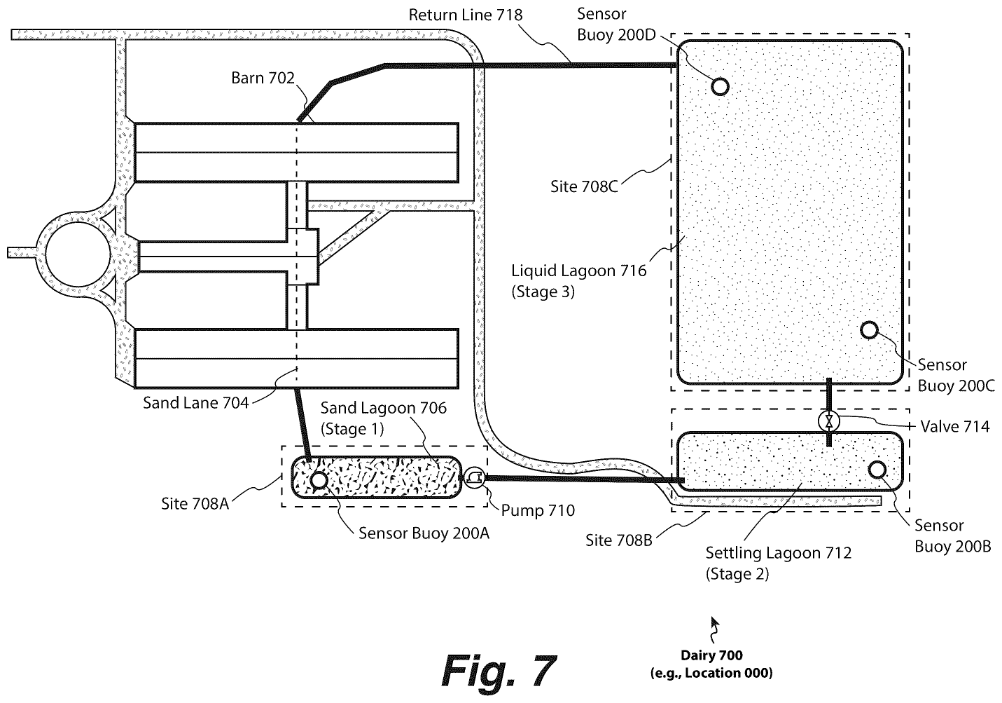

In one or more embodiments, the coordination server 300 may include a flow control engine 306 for controlling one or more water infrastructure devices 160. The flow control engine 306 may enable the coordination server 300 to automatically control hydraulic equipment such as valves, gates, and/or pumps, based on real-time water level measurements, predicted levels, and/or predetermined control thresholds. The flow control engine 306 may determine current water levels against configured control profiles 394 to determine when control instructions 396 should be generated and transmitted to the infrastructure device 160, for example to transfer water between interconnected lagoons to prevent overflow conditions and/or optimize capacity utilization across multiple bodies of water 100, for example as shown in conjunction with the embodiment of FIG. 6 and FIG. 7. When control thresholds are exceeded, as may be specified in the level 391 associated with the control profile 394, the flow control engine 306 may automatically generate and transmit control instructions 396 to designated infrastructure elements, which may enable rapid response to changing water conditions without manual intervention. Alternatively, proposed actions can be set for human confirmation (e.g., on the device 400). This automated control capability is particularly valuable for multi-lagoon wastewater treatment facilities, agricultural operations, and industrial water management systems where coordinated flow management between interconnected bodies of water is useful for preventing environmental incidents and/or maintaining operational efficiency.

In one or more embodiments, the flow control engine 306 may include computer readable instructions that when executed: receive water level data from multiple sensor buoys 200; query control profiles 394 containing threshold conditions and/or infrastructure response parameters; determine when current water levels exceed configured control thresholds (e.g., water levels, chemical levels, or other sensed thresholds); generate control instructions 396 specifying appropriate infrastructure responses such as valve operations or pump activation; and transmit control instructions 396 to designated infrastructure devices 160 through the network 150 to automatically initiate flow control actions based on real-time monitoring data.

In one or more embodiments, the coordination server 300 may include a data quality adjustment routine 308 for adjusting a frequency and/or quality of geospatial coordinates collected from sensor buoy 200.

The data quality adjustment routine 308 may be configured to dynamically adjust measurement parameters of the sensor buoy 200 based on real-time or predicted operational conditions and water level characteristics. The precision and/or data quality adjustment 308 may operate by continuously evaluating current water depth, volume per unit depth ratios, and/or proximity to critical thresholds to determine optimal data quality requirements 500 and data acquisition rates for each measurement cycle. When water levels are stable and volume changes per unit depth are minimal, data quality adjustment routine 308 may reduce data quality requirements and/or data collection frequency to conserve energy while maintaining adequate monitoring capabilities, for example by generating a remote procedure call to the data acquisition rate routine 228 of the sensor buoy 200 to configure data acquisition parameters. Conversely, as water levels approach critical thresholds or when volume changes more rapidly per unit depth (such as when shallow lagoons approach full capacity), the data quality adjustment routine 308 may automatically increase data quality requirements, coordinate determination rates (e.g., time resolution), and/or the quantity of geospatial measurements that may be averaged into a single geospatial coordinate 130 and/or geospatial data 260 reading to help ensure accurate detection of rapid changes and timely alert 392 generation. This adaptive approach provides significant advantages over fixed-precision monitoring systems by balancing measurement accuracy with energy efficiency based on actual operational needs, for example helping to extend autonomous operation time and/or conserve the power source 250 while ensuring critical conditions are likely to be detected with appropriate precision for environmental compliance and overflow prevention.

In one or more embodiments, the coordination server 300 may include a realtime alert system 309 for generating alerts 392 in response to one or more events. The realtime alert system 309 may be configured as a notification and/or warning system that may respond to periodically and/or continuously monitored water levels and generates immediate alerts when predetermined threshold conditions are exceeded (e.g., the overflow alert depth 120, the low alert depth 122, the prescribed maximum depth 116, the overflow depth 118, etc). The realtime alert system 309 may operate by receiving water level data from the level determination routine 304, comparing current measurements against configured alert profiles 390 containing threshold conditions for the body of water 100 associated with the site profile 350, and/or automatically generating and transmitting alert 392 notifications when critical conditions are detected. Alters 392 may also be generated based on depravities of depth or other levels, for example, change in depth over time, a rate of inflow 124 or outflow 126, and/or accelerations of depth or other levels.

When threshold violations are detected, the realtime alert system 309 may automatically generate alert 392 notifications that include relevant information about the current water level condition, the specific threshold that was exceeded, and the potential risk level associated with the condition. The system can generate different types of alerts including potential overflow alert 493 for imminent overflow conditions and low depth alert 491 for minimum operational level warnings.

The realtime alert system 309 may provide immediate notification capabilities by transmitting alerts 392 to designated recipients through multiple communication channels (e.g., text message, email, automated phone call, etc). Alert notifications may be sent to device 400 containing monitoring application 402, enabling operators (e.g., the user 170) to receive immediate notification of critical conditions on smartphones, tablets, and/or desktop computers.

The realtime alert system 309 may work in coordination with other system components to provide comprehensive monitoring and response capabilities. The system can integrate with the flow control engine 306 to automatically initiate infrastructure control actions when alert conditions are detected and/or alerts 392 are generated, enabling automated responses such as valve operations or pump activation to prevent overflow events and/or bodies of water 100 going dry. The realtime alert system 309 can also coordinate with the level projection engine 310 to generate predictive alerts 392 based on projected future water levels rather than waiting to arrive at actual thresholds, providing early prediction and warning capabilities that exceed traditional reactive alert systems. Such ability to generate multiple types of alerts based on different threshold conditions enables operators to implement a graduated response. Different alerts 392 may have different levels of urgency and/or appropriately urgent communication medium. For example, the current depth 114 arriving at the overflow alert depth 120 may initiate a text message, the current depth 114 reaching the prescribed maximum depth 116 may initiate an automatic phone call, and the current depth 114 reaching the overflow depth 118 may result in triggering multiple calls and text messages to several users 170, including potentially environmental regulators and/or remediation response teams.

In one or more embodiments, a realtime alert system 309 may include computer readable instructions that when executed determine (i) the depth of a body of water 100 (e.g., the wastewater lagoon) exceeds a threshold depth, and/or (ii) the volume of the body of water 100 exceeds a threshold volume; and generate an alert 392 (e.g., a potential overflow alert 493) that the body of water 100 exceeds the threshold depth and/or the threshold volume.

As just one example, the precipitation period 381 may be defined as each calendar month, while the level change 382 may be equated to a change in level during each precipitation period 381, together or apart from usage data. Although precipitation may be one of the most obvious factors, other aspects such as heat and wind periods may be similarly assessed, especially for broad and shallow bodies of water 100. In one or more embodiments, climate may be treated collectively and collapsed into a single consideration that determines level change 382 based upon all typical, historical, or average aspects of the specified period (e.g., average change each July).

The weather projection module 318 may be configured to provide short-term predictive capabilities by integrating real-time weather data and/or forecasts into water level projections. This may be particularly advantageous if the body of water 100 is near a critical point, such as the prescribed maximum depth 116, in which water 101 may need to be let out of the body of water 100 to create more capacity. The weather projection module 318 may receive weather forecast data including precipitation predictions, temperature forecasts, and/or other meteorological information that can affect water levels in the near term. The weather projection module 318 may analyze incoming weather events and estimate their impact on water levels, enabling generation of early warnings (e.g., the alert 392) for potential low water and/or overflow conditions caused by precipitation. Similarly, determination of an upcoming weather event, especially heavy precipitation, can be used as a trigger to automatically adjust precision requirements and/or monitoring frequency of the sensor buoy 200 in anticipation of relatively rapid weather-related water level changes through a call to the data quality adjustment routine 308.

In one or more embodiments, any predicted level change based on weather events may be deducted from any predicted level change attributable to the climate profile 380. As just one example, December precipitation may be light but consistent, such that any precipitation events are deducted off of expected precipitation due to climate either directly or proportionately. For instance, if December typically yields 4 inches of rainfall for the site, and 2.5 inches occurs by December 15th with no additional precipitation forecast, the remaining predicted rainfall attributable to climate (rather than specific weather forecasts) may be 2 inches (e.g., half of 4 for the remaining two weeks in December). In another example, monsoon rainfall in June may be heavy but uncertain: even rainfall exceeding what is predicted in the climate profile 380 may not negate all, most, or any of the rainfall specified in the climate profile 380 to ensure a conservative estimate of potential level increase. The user 170 may also be warned of unpredictability, or may be given boundaries for predicted level change based on historical lows and highs such that the user 170 is provided a statistical range of likely level change possibilities over one or more time horizons. Such potential level changes and/or statistical possibilities may be presented through a graphical user interface representation of the body of water 100 to help visual assessment.

Although water level is discussed with respect to usage, climate, and weather, values of other sensors 240 may be used to generating similar predictions. For example, nitrate concentrations may be tracked seasonally or monthly based on the interaction of agricultural runoff and crop fertilization periods. Similarly, oxygen levels of the water 101 may depend on algal blooms and water temperature which may vary based on month and season, each of which can be predicted as a climate and/or weather metric similar to water level.

In one or more embodiments, the coordination server 300 may include a usage profile 388, which may associate use periods 389 and/or use events 398 with level changes 399. In one or more embodiments, the coordination server 300 may include a usage profile 388, which may associate use periods 389 and/or use events 398 with level changes 399. The usage profile 388 may represent an operational data analysis system that enables the coordination server 300 to predict future water levels based on historical usage patterns and/or operational activities that affect water levels in the body of water 100.

The creation of usage profile 388 may be initiated with the coordination server 300 collecting and analyzing historical water level data alongside operational usage data for the body of water 100. This data collection process may include gathering information about regular operational activities such as wastewater discharge schedules, treatment process cycles, irrigation patterns, industrial process water usage, and maintenance activities that influence water levels. The data may be automatically gathered, inferred, and/or entered manually by the user 170. The usage profile 388 may incorporate temporal patterns in water usage, including daily operational cycles, weekly production schedules, seasonal usage variations, and special operational events that significantly impact water levels. This correlation analysis enables the usage projection routine 312 to quantify how different operational activities and usage patterns translate into specific water level changes for the particular body of water 100. Provided sufficient data, discrete effects or contributions of climate, weather, and/or usage may be able to be established.

In one or more embodiments, techniques known in the art from machine learning, deep learning, and/or artificial neural networks may be able to predict water level changes based in a number of inputs which have a known or suspected affect on water level, including operational data, weather data, climate data, sensor buoy 200 data, and/or other factors. Artificial neural network analysis may be particularly useful for receiving a weighting the impact of various simultaneous or near-in time factors. Training data from one or more locations, sites, and/or bodies of water 100 may be able to be used to train general predictive models usable as the level function 357 which can provide initial baseline estimates before more site-specified usage and historical data is gathered.

The usage profile 388 may enable the level projection engine 310 to generate predictive water level forecasts by applying current and/or planned operational activities to established usage correlation patterns. When operational schedules indicate upcoming activities that historically affect water levels, the usage projection routine 312 can estimate the expected water level changes and timeline, enabling the realtime alert system 309 to generate predictive alerts and/or the flow control engine 306 to initiate preventive control actions. The usage profile 388 may provide significant advantages for operational water level management by enabling the system to distinguish between normal operational variations and abnormal conditions requiring immediate attention, supporting proactive infrastructure control and/or capacity optimization based on anticipated rather than actual usage impacts.

In one or more embodiments, the usage projection routine 312 may include computer readable instructions that when executed: generate a level projection for a body of water 100 (e.g., a wastewater lagoon) based on inputs that include the water level data 370 based on at least one of the inflow rate 313, an outflow rate 314, and/or data within the usage profile 388 such as historical usage data; and determine a date in which a remaining capacity (e.g., the remaining capacity 110) of the body of water 100 is exceeded.

In one or more embodiments, the climate projection module 316 includes computer readable instructions that when executed: generate a climate profile 380 including average rainfall (e.g., for the location, site, or region of the body of water 100); associate a precipitation period with an increase in water level (e.g., wastewater level); and estimate an increase in the water level based in the climate profile 380. The level projection for the body of water 100 such as the wastewater lagoon may be based on inputs further including the increase in the water level based on the climate profile 380, according to one or more embodiments.

In one or more embodiments, the weather projection module 318 may include computer readable instructions that when executed determine occurrence of a precipitation event (e.g., rain, snow); associate the precipitation event with an increase in the water level of the body of water 100 such as the wastewater lagoon; receive weather forecast data; and/or estimate an increase in the water level based on the weather forecast data.

In one or more embodiments, the coordination server 300 may include an alert profile 390. The alert profile 390 may define standard or customized alerts based on one or more trigger conditions and deliver appropriate responses through various communication channels which may be specified in the alert profile 390. The alert profile 390 may be created and configured for each site and/or body of water 100 to account for unique operational requirements, environmental conditions, and/or regulatory compliance needs.

The alert profile 390 may include multiple types of trigger conditions that can initiate alert (e.g., defined as an alert action 393) generation at various levels 391. The alert action 393 may define the target(s) of the notifications and/or alerts, such as individual users 170 and/or their associated devices 400. Water level triggers may include depth thresholds such as the overflow alert depth 120, prescribed maximum depth 116, overflow depth 118, and/or low alert depth 122, enabling generation of alerts 392 at different stages and/or water levels. Volume-based and/or capacity-based triggers may utilize remaining capacity 110 percentages or absolute volume measurements to provide early warning when storage capacity approaches specified limits. The alert profile 390 may also incorporate sensor-based triggers from additional sensors 240, including chemical concentration thresholds from chemical sensor 242, pH level violations from pH sensor 244, temperature extremes from thermometer 246, and/or weather condition alerts from weather sensor 248. Predictive triggers may be based on level projections from the level projection engine 310, enabling generation of alerts 392 based on anticipated future conditions.

The alert profile 390 may define graduated response mechanisms that escalate based on the severity and/or urgency of detected conditions. Low-priority alerts 392 such as routine monitoring notifications may be transmitted via email or logged for later review. Medium-priority alerts such as approaching capacity warnings may trigger text message notifications to designated operators and/or facility managers. The alert profile 390 may also coordinate with external systems such as Supervisory Control and Data Acquisition networks (SCADA networks), environmental monitoring databases, and regulatory reporting systems to ensure comprehensive documentation and compliance reporting. High-priority alerts such as imminent overflow conditions may initiate immediate phone calls, multiple simultaneous notifications to emergency response teams, and automatic activation of backup communication systems. Critical alerts such as overflow depth 118 violations may trigger comprehensive emergency response protocols including notifications to environmental regulators, emergency services, and/or remediation response teams. In addition, automatic failsafes may be initiated, as further described in conjunction with the control profile 394, according to one or more embodiments.

In one or more embodiments, the coordination server 300 may include a control profile 394. The control profile 394 may define automated infrastructure control parameters and responses based on water level conditions (e.g., as determined through the geospatial data 260 and/or water level data 370) and/or sensor 240 measurements, enabling initiation of automatic control actions such as valve operations, pump activation, and/or other flow management operations without manual intervention. The control profile 394 may be created and configured for each site and/or body of water 100 to account for unique operational requirements, infrastructure capabilities, and/or safety protocols.

The control profile 394 may include multiple types of trigger conditions that can initiate automated control responses at various levels 391. Water level triggers may include depth thresholds such as the overflow alert depth 120, prescribed maximum depth 116, and critical capacity percentages that require immediate infrastructure response to prevent overflow conditions. Volume-based triggers may utilize remaining capacity 110 calculations to automatically initiate water transfer between interconnected bodies of water 100 (such as multi-stage lagoons) when capacity limits approach various levels. The control profile 394 may also incorporate sensor-based triggers from additional sensors 240, including chemical concentration thresholds that may prevent discharge when contaminant levels exceed safe limits, pH level violations that require treatment system activation, and temperature extremes that may trigger cooling or heating system responses. Predictive triggers may be based on level projections from the level projection engine 310, enabling preventive control actions based on future conditions.

The control profile 394 may define graduated control responses that escalate based on the severity and urgency of detected conditions. A control action 395 may be paired with a level 391, whether a water level or other level sensed through one or more sensors 240 (e.g., a chemical concentration). The control action 395 may define the target(s) of control instructions 396, such as infrastructure devices 160 connected to the coordination server 300 over the network 150. In one or more embodiments, the control instruction 396 may include a pump control instruction 397A and/or a valve control instruction 397B. Low-priority control actions such as routine flow adjustments may trigger automatic valve position changes to optimize capacity distribution between bodies of water 100. Medium-priority control actions such as approaching capacity warnings may activate pump systems to transfer water from bodies of water 100 nearing capacity to those with available capacity. High-priority control actions such as imminent overflow conditions may initiate emergency pump activation, automatic valve opening for rapid discharge, and/or coordinated multi-site flow management to prevent environmental incidents or infrastructure damage, such as wash out of an earthen berm 106. Critical control actions such as overflow depth 118 violations may trigger comprehensive emergency response protocols including automatic activation of all available discharge systems, emergency pump deployment, and failsafe mechanisms that prioritize environmental protection over operational efficiency. In one or more embodiments, it will be noted that control profile 394 may issue instructions for a user 170 to perform one or more manual operations such as opening a gate or valve that is not automated and/or connected through the network 150.

The provisioning application 320 may represent a comprehensive sensor buoy management system that may enable the coordination server 300 to efficiently deploy, configure, and assign sensor buoys 200 to specific bodies of water 100 throughout the monitoring network. The provisioning application 320 may serve as the central management interface for establishing and maintaining the operational relationships between sensor hardware, site profiles, and monitoring requirements across multiple bodies of water and facilities. The provisioning application 320 may also enable adaptation of the sensor buoy 200 to allow for rapid setup, deployment, and customization at new bodies of water 100. For example, in one or more embodiments, the device 400 may connect directly with the controller 220 (e.g., through Bluetooth®), which may allow the device 400 to act as an interface to setup, configure, and/or provision the sensor buoy 200 at the body of water 100. The setup may include linking the sensor buoy 200 with an existing account and/or the site profile 350, according to one or more embodiments.

The provisioning application 320 may operate by maintaining and/or managing an inventory of available sensor buoys 200, each of which may be identified by their unique buoy UID 201, along with their current operational status, battery levels, calibration dates, and/or deployment history. When a new body of water 100 requires monitoring or when existing monitoring needs change, the provisioning application 320 may facilitate the selection and assignment process by matching sensor buoy capabilities with site-specific monitoring requirements and/or reconfigure the sensor buoy 200 for a new operational context. The user 170 may consider factors such as the body of water's size, expected precision requirements, environmental conditions (such as corrosive chemicals that might affect tether 214 materials), communication network availability, and power generation potential when sensor buoys 200 for deployment.

In one or more embodiments, the provisioning application 320 may manage the assignment process by updating site profiles 350 to include references to assigned sensor buoys 200 through the assigned sensors 354 attribute, which may store buoy references 356 containing the buoy UID 201 of each deployed sensor buoy 200. This creates the operational linkage that enables the coordination server 300 to associate incoming geospatial data 260 from specific sensor buoys 200 with their corresponding bodies of water 100 and site-specific level functions 357. The provisioning application 320 may also configure initial operational parameters for newly assigned sensor buoys 200, including wake routine 224 schedules, data acquisition rate routine 228 settings, and/or precision requirements 500 based on the specific monitoring needs of each body of water 100.

In one or more embodiments, the provisioning application 320 may provide ongoing management capabilities including sensor buoy 200 health monitoring, maintenance scheduling, and redeployment coordination when sensor buoys 200 require service or when monitoring priorities change. The provisioning application 320 may track sensor buoy performance metrics, battery life, solar panel 210 efficiency, and communication reliability to optimize deployment strategies and predict maintenance needs. When sensor buoys 200 require replacement or repositioning, the provisioning application 320 may facilitate the decommissioning and/or reassignment process by updating site profiles 350 and ensuring continuity of water level monitoring during transition periods.

The provisioning application 320 provides significant advantages for large-scale water monitoring operations by enabling centralized management of distributed sensor networks, optimizing sensor buoy 200 utilization across multiple sites, and ensuring appropriate matching of monitoring capabilities and configuration with site-specific requirements. The provisioning application 320 may support scalable deployment strategies that can accommodate growing monitoring networks while maintaining operational efficiency and data quality standards essential for environmental compliance and overflow prevention across diverse water management applications.

In one or more embodiments, locations, sites, and/or sensor buoys 200 may be related and/or associated within a data structure, including the formation of operational hierarchies. For example, a location may specify an area or facility having an overall function and which may have multiple sites, for example a mine, dairy, and/or municipal water treatment facility. In one or more embodiments, a location profile 330 may be set up to model a location, which may include and/or reference one or more site profiles 350 each set up to model an area including one or more bodies of water 100. Each site profile 350 may include and/or reference one or more sensor profiles 340 representing a sensor buoy 200 or other sensor array.

In one or more embodiments, the location profile 330 may include a unique identifier 331 of the location (e.g., the UID 331, which may also be referred to as the location UID 331), a location name 332 (e.g., a name of the facility, a street address), and/or an attribute storing associated sites 333 which may include a list of references to associated sites as the site reference 334 (abbreviated site ref. 334 in FIG. 3), each of which may store as a value the unique identifier 351 of the site profile 350.

In one or more embodiments, the site profile 350 may include a unique identifier 351 of the site profile 350 (e.g., the UID 351, which may also be referred to as the site UID 351), a site name 352 (e.g., “lithium evaporation pond 22”), and an attribute storing references to assigned sensors, the assigned sensors 354 attribute. The assigned sensors 354 may store a set of references to sensor buoys 200, for example with a buoy reference 356 attribute each storing as a value an instance of the buoy UID 201.

The site profile 350 may include or reference to one or more level functions 357. A level function 357 may receive an input that includes the elevation coordinate 136 to determine a level of the body of water 100. The level may include a depth (e.g., the current depth 114), a volume (e.g., a current volume), a spatial extent of the water (e.g., a “footprint” of the water 101 or its edge), a capacity (e.g., the used capacity 108), and/or other measure or metric of the amount of water 101 in the body of water 100. In one or more embodiments, determination of one type of level may result in automatic determination of other levels. For example, depth and/or volume may be used to directly determine used capacity 108 and/or remaining capacity 110. The level functions 357 may be created through a variety of means, including without limitation through: (i) sole use of engineering plans or specifications (e.g., description of a large cylindrical concrete water cistern); (ii) field-verified measurements in combination with engineered plans (e.g., confirming an elevation of a berm 106 and using wall and slope information from engineered plans to build an approximate model of a lagoon); and/or (iii) gathering complete topographic data for an area, for example by walking a pattern to generate a grid and/or matrix of geospatial coordinates 130 defining a topology or surface contour for the body of water 100. In one or more embodiments, the level functions 357 may include additional data or modeling to account for expected filling with solids, sedimentation, and/or other aspects which can affect total capacity 112.

In one or more embodiments, the site profile 350 may include the site data object 360, which may include geospatial data that defines the site and/or the body of water 100. The site data object 360 may include specification data 367, which may be gathered from field measurements, survey data, engineered plans, permit specifications, and/or manually entered approximations or estimates. For example, the site data object 360 may include a maximum depth data 368 specifying the prescribed maximum depth 116, and/or the slope specification data 369 which may specify the wall slope 105 and/or other characteristics of the wall 104.

As also previously shown and described in conjunction with FIG. 2, the site data object 360 may further include geospatial descriptions of one or more features 362 of the site and/or the body of water 100. For example, the feature 362 may include polygon data 364 for a perimeter (e.g., the perimeter contour data 142, the bottom contour data 144, an area of potential flooding or inundation for the site if the body of water 100 overflows, etc.). In one or more embodiments, the feature 362 may also include a set of contour data 366 which may describe a two dimensional or three dimensional contour with elevation data from which a three dimensional shape can be generated, deduced, and/or inferred. As just one example, and referring to FIG. 1C, the bottom contour data 144 may be used to both define a perimeter of the floor 102 of the body of water 100, but also may be used to define a topological plane. This may be useful, for example, where the floor 102 is sloped such that the body of water 100 has a shallow end and a deep end but is level across one direction or axis, similar to a common residential swimming pool design. As shown and described throughout the present embodiments, the site data object 360 and data thereof may be used to define the level functions 357, according to one or more embodiments.

Once positioned, acquired operational data for the body of water 100 may be stored in or in association with the site profile 350, according to one or more embodiments. The site profile 350 may store the data as the water level data 370, which may include attributes such as the date 371, the time 372, the geospatial coordinate 130 (which may be or include the corrected geospatial coordinate 134), an elevation coordinate 136, and/or attributes for calculated levels such as volume 374 and/or the depth 376. Additional data from the sensors and/or sensor buoy 200 may be stored, for example status messages from the controller 220, battery health indicators, solar output, and/or other useful metrics.

It should be noted that, in one or more embodiments, non-elevation portions of the geospatial coordinate 130 may still be useful. For example, the sensor buoy 200 may be free-floating, and failure of any movement in the x-y direction may indicate the sensor buoy 200 has been beached or is no longer in the water 101. In another example, a geofence may be established, the breaching of which may indicate that the sensor buoy 200 has become untethered (e.g., the tether 214 is broken) or a strong wind or current has pushed the sensor buoy 200 and/or anchor 216 outside of a designated area, which may reduce or compromise its effectiveness in determining water level.

The climate profile 380 may represent an environmental data analysis system that enables the coordination server 300 to predict future water levels based on long-term climate patterns and/or seasonal weather trends. The climate profile 380 may be created through systematic analysis of historical precipitation data, seasonal rainfall patterns, snowmelt patterns, and/or regional climate characteristics that influence water level changes in the body of water 100 over extended periods.

The creation of climate profile 380 may be initiated with the coordination server 300 collecting and analyzing historical weather data for the geographic region embracing the body of water 100. Such climate data collection process may include querying precipitation records from meteorological databases, regional weather stations, and/or climate monitoring networks to establish baseline precipitation patterns over multiple years or decades. The climate profile 380 may incorporate seasonal variations in rainfall, including wet and dry seasons, average monthly precipitation totals, and historical precipitation extremes that can significantly impact water levels in lagoons, reservoirs, and treatment facilities.

In one or more embodiments, the climate profile 380 may establish correlations between precipitation periods 381 and corresponding level changes 382 by analyzing historical water level data and/or usage data alongside weather records. This correlation analysis enables the climate projection module 316 to quantify how different precipitation periods (e.g., the precipitation period 381) equate into water level changes (e.g., the level change 382) for the specific body of water 100, accounting for factors such as watershed characteristics, catch basin size, surface runoff patterns, and/or absorption rates. The climate profile 380 may include seasonal adjustment factors that account for varying evaporation rates, temperature effects, and other environmental conditions that influence the relationship between precipitation and water level changes throughout the year. Once established, the climate profile 380 may enable the level projection engine 310 to generate predictive water level forecasts by applying historical climate patterns to current conditions. The climate profile 380 can provide significant advantages for long-term water level management by enabling the system to distinguish between normal seasonal variations and abnormal conditions that may require immediate attention.

The weather profile 384 may represent a relatively short-term predictive analytics system (e.g., based on a 1, 3, 7, and/or 10 day forecast) that enables the coordination server 300 to anticipate immediate water level changes based on specific weather events and/or real-time meteorological conditions. The weather profile 384 may be created through systematic integration of real-time weather data sources, weather forecast services, and historical correlation analysis between specific precipitation events and corresponding water level increases in the body of water 100.

The creation of weather profile 384 may be initiated with the coordination server 300 establishing connections to meteorological data sources including national weather services, regional weather monitoring stations, and/or commercial weather forecast providers. The weather profile 384 may incorporate real-time precipitation data, temperature readings, humidity levels, and/or wind conditions that can influence water level changes through direct precipitation, evaporation rates, and runoff patterns. The system may continuously monitor current weather conditions and receive forecast data for the geographic area embracing the body of water 100, enabling the weather projection module 318 to anticipate short-term water level changes.

In one or more embodiments, the weather profile 384 may establish correlations between specific precipitation events 385 and corresponding level changes 386 by analyzing actual weather data alongside concurrent water level measurements from the sensor buoy 200. This correlation analysis enables the weather projection module 318 to quantify how different types of precipitation events (such as light rain, heavy downpours, or extended storm systems) translate into specific water level increases for the particular body of water 100. The weather profile 384 may account for factors such as precipitation intensity, duration, temperature effects on absorption rates, and seasonal variations in ground saturation that influence the relationship between weather events and water level changes.

The weather profile 384 may enable the level projection engine 310 to generate predictive water level forecasts by applying current weather conditions and forecast data to established correlation patterns. When weather forecast data indicates incoming precipitation events, the weather projection module 318 can estimate the expected water level increase and timeline, enabling the realtime alert system 309 to generate predictive alerts before actual thresholds are expected. The weather profile 384 may provide significant advantages for immediate water level management by enabling the system to distinguish between normal weather-related variations and abnormal conditions requiring immediate attention, supporting proactive infrastructure control and overflow prevention based on anticipated rather than actual weather impacts.

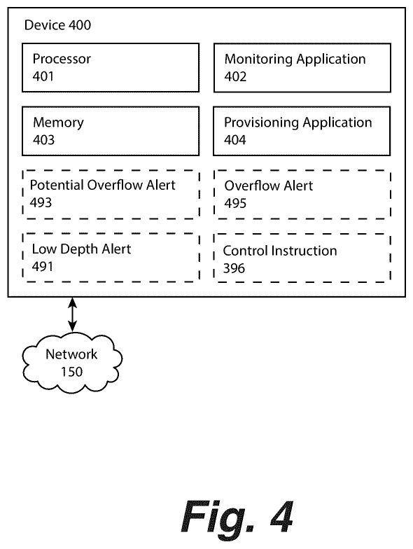

FIG. 4 illustrates a block diagram of device 400 including a processor, memory, and monitoring application 402 usable by the user 170 to monitor and/or control the water monitoring system 190, according to one or more embodiments. The device 400 comprises processor 401 for executing application software and memory 403 for storing operational programs and/or data. The primary user interface is provided through monitoring application 402, which enables operators to receive alerts, view system status, and manage monitoring parameters. The monitoring application 402 may be a desktop software application and/or a mobile application, according to one or more embodiments. The monitoring application 402 serves as the primary interface between users and the coordination server 300, providing real-time access to water level data, level analytics, level predictions, alert notifications, and system control capabilities.

The device 400 connects to the coordination server 300 through network 150, enabling communication with coordination server 300 and receiving data from multiple sensor buoys 200 deployed across various bodies of water 100. This network connectivity allows the device 400 to receive real-time alerts such as potential overflow alert 493, low depth alert 491, and control instructions 396 generated by the coordination server 300 based on current water level conditions. Undeliverable alerts may be stored and re-sent when the device 400 reconnects to the network 400.

In one or more embodiments, the monitoring application 402 may provide comprehensive interface capabilities for system management and monitoring. Users 170 can configure monitoring parameters, view historical water level data, receive automated alerts when threshold conditions are exceeded, and monitor the operational status of deployed sensor buoys 200. The monitoring application 402 may enable remote management of the location wide water level monitoring infrastructure from a centralized interface, supporting both individual site monitoring and multi-site facility management.

Although one instance of the device 400 is shown in FIG. 1A, it will be appreciated that many instances may be associated with a single instance of the water monitoring system 190, such as a few, tens, hundreds, thousands, or more depending on the size and complexity of the location and its sites.

FIG. 5 illustrates a cross-sectional view of sensor buoy 200 with a data quality requirement 500 visually represented, according to one or more embodiments. FIG. 5 illustrates a change in time resolution, precision, and/or accuracy required as the body of water 100 rises, especially as volume per unit depth and/or the rate of inflow 124 increases. For example, the gradations of the data quality requirement 500 in FIG. 5 may represent equal gradations of volume, according to one or more embodiments. In one or more other embodiments, the data quality requirement 500 may be primarily a requirement based on increased accuracy and/or precision as the sensor buoy 200 reaches the prescribed maximum depth 116 and/or the overflow depth 118, rather than related to volume and/or remaining capacity 110.

The data quality requirement 500 may represent adaptive measurement precision and/or accuracy that may adjust based on operational conditions. In another example, the data quality requirement 500 may be dynamically adjusted based on predicted inflows 124. When water levels are low and volume changes per unit depth are minimal and/or when substantial inflow is unlikely, the system can operate with reduced data quality requirement 500 to conserve energy while maintaining adequate monitoring capabilities. As water levels increase and approach critical thresholds, the system can automatically increase data quality requirements to ensure accurate detection of rapid changes and potential overflow conditions.

The data quality requirement 500 can adjust in several aspects. First, more frequent geospatial data 260 can be determined, logged, and/or transmitted (e.g., resulting in more instances of the water level data 370 on the coordination server 300) to result in increased time resolution. Second, an accuracy and/or precision of the geospatial data 260 and/or resulting water level data 370 can be increased, for example by taking longer geospatial readings from the geospatial positioning unit 208, waiting for better or more consistent spatial correction data, averaging more geospatial coordinates 130, through more stringent precision filtering, and/or other techniques known in the art for processing geospatial data.

In one or more embodiments, the data quality requirement 500 may be specified through use of one or more of the level functions 357, including with reference to any of the alert profiles 390 and/or control profiles 394. For example, the volume function 359 may be used to determine the data quality requirement 500 for each expected rise in elevation of the water 101.

The adjustable and/or dynamic data quality requirement 500 may provide advantages over traditional fixed-precision monitoring systems by optimizing measurement accuracy based on actual operational needs. During low-risk periods and/or stable water levels, the system may conserve energy through reduced data quality requirements. During high-risk periods approaching overflow conditions, the system can automatically increase precision to ensure accurate detection and timely alert generation. This intelligent adaptation enables long-term autonomous operation while maintaining the high accuracy necessary for operational efficiency, environmental compliance, and/or overflow prevention.

FIG. 6 illustrates a hydraulic coupling system between two bodies of water 100 (e.g., lagoon 600A and lagoon 600B) each monitored by a sensor buoy 200 (e.g., sensor buoy 200A and sensor buoy 200B), according to one or more embodiments. FIG. 6 demonstrates how water level monitoring may enable coordinated management of interconnected bodies of water 100 through automated flow control based on water level measurements from multiple sensor buoys 200, including real-time water level measurement.

It should be noted that in some cases, volume per unit depth may be more important to determine. For example, where a minimum amount of water 101 must be maintained in the body of water 100, the sensor buoy 100 may increase time resolution as the body of water 100 empties. Otherwise, where a constant rate of removal is maintained, there may be a sudden drop in depth of the water as the deeper areas with lower volume holding capacity are emptied. In such case, the data quality requirement 500 as shown in FIG. 5 may be inverted.

Specifically, FIG. 6 illustrates two interconnected lagoons: lagoon 600A and lagoon 600B, which are connected through hydraulic coupling 602 such as a pipe, culvert, canal, or other water conveyance system. Each lagoon 600 may be equipped with its own sensor buoy 200 (e.g., sensor buoy 200A and sensor buoy 200B, respectively), providing independent water level monitoring capabilities for individual and/or coordinated location management. The automatic valve 604 may be opened, manually and/or by receiving a control instruction 396. The automatic valve 604 is an instance of the hydraulic infrastructure device 160, according to one or more embodiments.

The automatic valve 604 can be controlled remotely based on water level data received from the sensor buoys 200A and/or the sensor buoy 200B. Flow can be automatically initiated from lagoon 600A to lagoon 600B when sensor buoy 200A detects appropriate conditions. For example, it may be advantageous (or required by regulation) to primarily use lagoon 600A for runoff, and only use lagoon 600B as an overflow backup system. Conversely, in another example, lagoon 600A may be intended to remove a certain chemical (e.g., sulfuric acid from a former mining site), and water 101 may be intended to freely overflow to lagoon 600B. However, under certain conditions, such as if the pH drops too far (e.g., as measured by the pH sensor 244), which may indicate failure of sulfuric acid removal, the automatic valve 604 may automatically close. In such case, contractors or regulators may be notified such that additional lime or other remediation chemical can be added to lagoon 600A. In yet another example, lagoon 600A may receive discharges of sediment-laden liquid, and sensor buoy 200A may determine turbidity, water clarity, and/or dissolved solids. Upon dropping to environmentally permissible levels (e.g., the sediment has settled on the floor 102 of lagoon 600A), the coordination server 300 may automatically open the automatic valve 604. Similarly, if lagoon 600A is about to overflow, the coordination server 300 may be able to activate a different pump (not shown) which may include a sediment filter to increase the remaining capacity 110.

One skilled in the art of water management will appreciate additional applications based on the real time, high accuracy, high precision, and/or predictive capability of the water monitoring system 190 and components thereof.

FIG. 7 illustrates a working example of a multi-lagoon wastewater treatment facility with interconnected sensor buoys 200 and control systems at a large scale dairy, according to one or more embodiments. FIG. 7 may demonstrate a comprehensive wastewater treatment operation that utilizes multiple interconnected lagoons with coordinated water level monitoring and/or automated flow control capabilities, according to one or more embodiments. The dairy 700 may be represented by a location profile 330. The location of FIG. 7 includes multiple monitoring sites (site 708A, site 708B, and site 708C) that correspond to different modes of the treatment facility, each with dedicated sensor buoys 200 for comprehensive coverage. The sites are shown interconnected through hydraulic infrastructure including the pump 710 and the valve 714, and various hydraulic coupling lines which may be pipes, canals, or other waterways.

The dairy 700 may include a barn 702 that includes a sand lane 704 as part of the operational infrastructure for removing cow waste and sand bedding from the sleeping quarters of the dairy 700. Water and sand mixture may be washed down the sand lane 704, moving via simple gravitational flow, and into the sand lagoon 706. The sand lagoon 706 may be used for recovery, washing, and drying of sand for reuse. The sand lagoon 706 and its description may be defined as site 708A, which may have an associated site profile 350A. Site 708A may include a pump 710 which may pump liquid out of a low end of the sand lagoon up to the settling lagoon 712. In one or more embodiments, a sensor buoy 200A may be placed at one end of the sand lagoon 706 (e.g., the low end) such that the water level can be determined. If the water level is too high, alerts can be generated and/or the pump 710 automatically activated to move liquid to the settling lagoon 712, according to one or more embodiments.

The settling lagoon 712 may serve as a secondary treatment stage in the multi-lagoon wastewater treatment facility, receiving liquid discharge from the sand lagoon 706 via pump 710. The settling lagoon 712 may be specifically designed for gravitational separation and removal of suspended solids, including fine manure particles and excess small grain sand that were not captured during the initial sand separation process in the sand lagoon 706. The settling lagoon 712 may operate through extended retention time that allows heavier particles to settle to the floor 102 while lighter organic matter and dissolved nutrients remain in suspension for further treatment.

The settling lagoon 712 may be equipped with sensor buoy 200B to continuously monitor water levels and/or ensure optimal settling conditions. The sensor buoy 200B may enable the coordination server 300 to track the accumulation of settled solids over time and determine when the settling lagoon 712 requires maintenance such as solids removal or when capacity approaches limits that could compromise treatment efficiency. The settling lagoon 712 may include valve 714 that can be automatically controlled based on water level data from sensor buoy 200B to regulate discharge to the liquid lagoon 716, ensuring that only adequately settled liquid proceeds to the final treatment stage. As one example, depth per unit volume may be able to be historically tracked to known inflow from the pump 710 and/or measured outflow from the valve 714, created volumetric flow data that when equated with depth can be used to determine sedimentation (and corresponding loss of capacity) over time.

The water level monitoring system may provide critical operational advantages for the settling lagoon 712 by enabling precise control of retention time, which may directly affect settling efficiency. When sensor buoy 200B detects water levels approaching capacity limits, the coordination server 300 can automatically activate valve 714 to discharge clarified liquid to the liquid lagoon 716, maintaining optimal settling conditions while preventing overflow events. The system may also monitor settling rates by tracking water level changes over time, providing valuable data for optimizing treatment processes and predicting maintenance requirements for solids removal operations.

The liquid lagoon 716 may serve as the final treatment stage in the multi-lagoon wastewater treatment facility, receiving clarified liquid discharge from the settling lagoon 712 via valve 714. The liquid lagoon 716 may be specifically designed for long-term biological treatment and polishing of liquid runoff, providing extended retention time that allows for advanced nutrient removal, pathogen reduction, UV and oxygen exposure, UV and oxygen exposure, and final clarification before water reuse or discharge.

The liquid lagoon 716 may operate through extended biological processes including aerobic and anaerobic treatment that further reduces organic matter, nitrogen compounds, and phosphorus levels in the liquid effluent. The liquid lagoon 716 may utilize natural biological processes such as algae growth, bacterial decomposition, and settling to achieve final treatment objectives. The liquid lagoon 716 may be equipped with sensor buoy 200C to continuously monitor water levels, ensuring optimal treatment conditions and preventing overflow events that could compromise treatment efficiency or environmental compliance. Similarly the liquid lagoon 716 may include a sensor buoy 200D near the return line 718 which may include sensors usable to determine whether the liquid is usable in the return line 718 for reuse to wash the sand lane 704 of the barn 702.

The liquid lagoon 716 may include return line 778 that enables treated water to be recirculated back through the treatment system for reuse in dairy operations such as barn washing, equipment cleaning, and/or irrigation applications. This water reuse capability may provide significant operational advantages by reducing freshwater consumption, minimizing discharge volumes, and creating a closed-loop treatment system that maximizes resource recovery. The return line 778 may be controlled based on water quality measurements from sensor buoy 200D and/or additional water quality sensors, ensuring that only adequately treated water is recirculated for reuse applications.

The water monitoring system 190 instantiated for the dairy 700 may provide critical operational advantages for the liquid lagoon 716 by enabling precise control of retention time and treatment efficiency. The system may also coordinate with upstream lagoons (e.g., the sand lagoon 706 and the settling lagoon 712) through automated valve and pump control to balance treatment loads across the entire facility, ensure consistent treatment performance and preventing system overload during peak discharge periods. In the present example, sensor buoys 200 may be easily deployed and configured. Correction data may be provided through LTE and/or 5G cellular network, including access to municipal or department of transportation correction data servers (alternatively, a local base station may be used with radio or other wireless connectivity to each sensor buoy 200). One or more farmers, maintenance personnel, and/or regulators (each users 170) may have access to information about the dairy 700 and its lagoon remediation system through the monitoring application 402 on the device 400. In one or more embodiments, the device 400 may be a desktop computer and the monitoring application 402 may offered as a web portal through a browser application and/or web app.

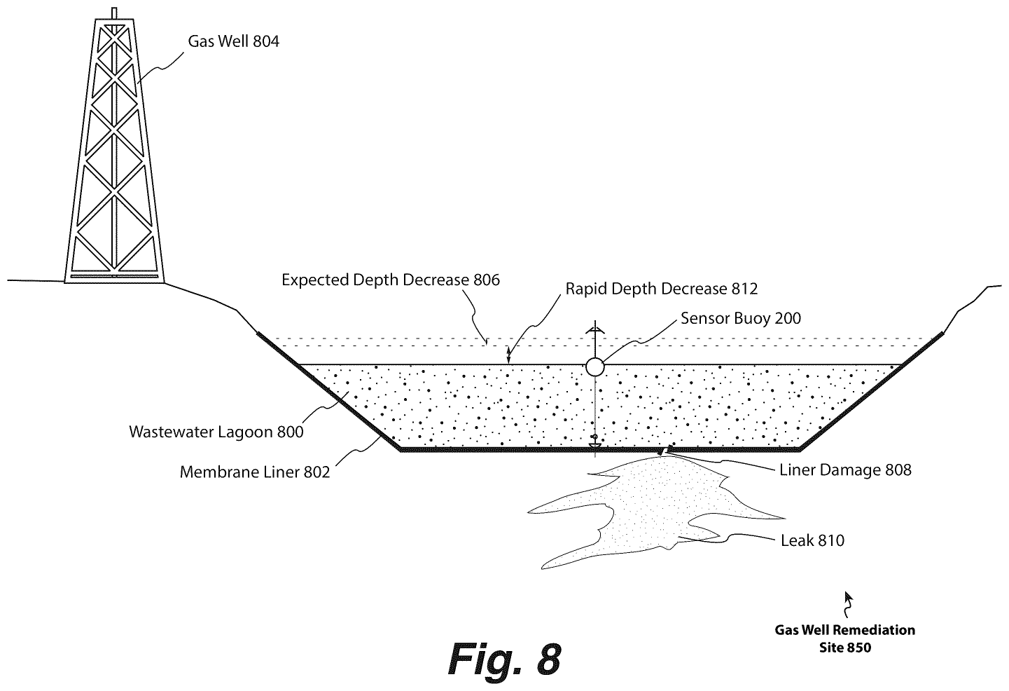

FIG. 8 illustrates a gas well remediation site 850 illustrating a working embodiment in which the sensor buoy 200 may be used to monitor a wastewater lagoon 800, including detecting an unexpected decrease in the level of the wastewater lagoon 800 indicative of a leak 810 in a membrane liner 802, according to one or more embodiments.

Wastewater from hydraulic fracturing operations, commonly known as produced water or flowback water, may represent a complex mixture of chemicals and/or contaminants that requires specialized containment and treatment. This wastewater may contain high concentrations of total dissolved solids (TDS), heavy metals such as barium and strontium, naturally occurring radioactive materials (NORM) such as thorium or radon, volatile organic compounds (VOCs), and various chemical additives used in the fracturing process including biocides, corrosion inhibitors, and/or friction reducers. The wastewater may also contain high levels of chlorides and other salts, making the wastewater significantly more saline than seawater, along with hydrocarbons, suspended solids, and/or potentially toxic substances that pose environmental and health risks if not properly managed. Due to its complex composition and high contamination potential, hydraulic fracturing wastewater may require secure containment in lined lagoons to prevent groundwater contamination, surface water pollution, and soil degradation, making precise water level monitoring and leak detection capabilities advantageous for environmental protection and regulatory compliance at gas extraction sites.

FIG. 8 illustrates a working example of a gas well remediation site 850 that shows wastewater lagoon 800 with membrane liner 802 that may serve as a containment barrier for wastewater generated during gas extraction operations. One or more gas wells 804 may be positioned adjacent to the lagoon, representing the primary extraction infrastructure that generates wastewater requiring quarantining and/or treatment. The membrane liner 802 may serve an important role by preventing wastewater from contaminating surrounding soil and groundwater.

FIG. 8 demonstrates how the water monitoring system 190 can be deployed at gas well extraction sites (e.g., hydraulic fracturing sites) to monitor wastewater lagoon levels 800 and predict levels (e.g., as shown and described throughout the present embodiments). In addition, FIG. 8 demonstrates use of the sensor buoy 200 to detect leaks, unintended or unexpected inflow (e.g., the inflow 124) or outflow (e.g., the outflow 126), including as a result of damage to the wastewater lagoon 800 such as membrane damage 808 to the membrane liner 802, what may be advantageous environmental monitoring for gas extraction operations.

The coordination server 300 may process water level data from sensor buoy 200 and concurrent measurements against expected operational patterns (e.g., stored within the usage profile 388). When a rapid depth decrease 812 is detected that cannot be attributed to normal operational factors such as evaporation or controlled discharge, an alert 392 may be generated indicating the unexpected drop in water level, for example as a result of a stuck valve, leaking infrastructure, or as presently illustrated membrane liner damage 808. This early detection capability may enable rapid response to prevent environmental contamination and ensure compliance with environmental regulations governing gas well operations.

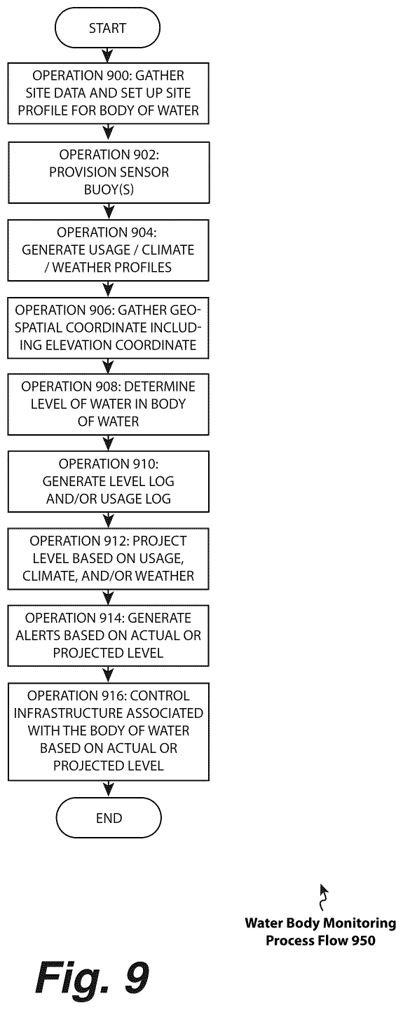

FIG. 9 illustrates a water body monitoring process flow 950, according to one or more embodiments. FIG. 9 demonstrates an operational method for various aspects of the water monitoring system 190, including continuous monitoring, predictive analytics, and automated control capabilities for bodies of water 100 such as wastewater lagoons and treatment facilities, according to one or more embodiments.

The water body monitoring process flow 950 may begin with operation 900 which may gather site data (e.g., within the site data object 360) and set up the site profile 350 for the site embracing and/or including the body of water 100. This initial setup phase may establish the foundational parameters necessary for accurate water level monitoring and volume calculations. In one or more embodiments, provisioning may take place on site, for example via a Bluetooth link between the device 400 (e.g., such as a smartphone) and the sensor buoy 200. The user 170 may log into an online account and associated the sensor buoy 200 with an existing deployment of the water monitoring system 190 for which the user is permissioned and/or an administrative user.

Operation 902 provisions one or more sensor buoys 200 for deployment in the designated body of water 100. Operation 902 may assign sensor profiles 340 to the site profile 350, for example, the assigned sensors 354 attribute. Operation 902 may also configure and/or calibrate the one or more sensor buoys 200.

The method may generate one or more operational profiles through operation 904, which may create usage profiles 388, climate profiles 380, and/or weather profiles 384 that may be used to enable predictive monitoring capabilities. These profiles may incorporate and generate predictive mathematical functions and historical data, seasonal patterns, and/or environmental factors that influence water level changes. Operation 906 may initiate continuous geospatial coordinate 130 gathering including elevation coordinate 136 data from the deployed sensor buoy 200, providing real-time positioning and elevation information usable for accurate water level determination.

Operation 908 may determine the level of water in body of water 100 using the geospatial coordinates 130, including the elevation coordinate 136, in combination with the pre-configured and/or dynamically adjustable site profiles 350. In one or more embodiments, the elevation coordinate 136 may be input into the depth function 358 and/or volume function 359 to calculate current water levels and/or remaining capacity 110. Operation 910 generates level logs and/or usage logs that maintain historical records of water level changes, supporting both operational management, improved predication, and/or regulatory compliance requirements. The level logs may be stored as the water level data 370 for a particular date 371 and time 372.

The method may provide predictive water level capabilities through operation 912, which may project future water levels based on usage patterns, climate data, and/or weather forecasts. This predictive analysis may enable early identification of potential overflow conditions (e.g., usable to result in generation of alerts 392) and/or supports proactive management decisions (e.g., usable to result in generation of one or more control instructions 396).

Operation 914 may generate alerts 392 based on both current water levels and/or projected levels. Alerts 392 based on projected levels may also be used to provide early warning capabilities that exceed traditional threshold-based alert systems.

Operation 916 may control infrastructure associated with the body of water 100 based on actual and/or projected water levels. This automated control capability may enable preventive actions such as valve operations, pump activation, and/or water transfer between connected bodies of water 100 such as wastewater lagoons to prevent overflow events and/or optimize capacity utilization across multiple bodies of water 100.

This water body 100 monitoring process may provide significant advantages over traditional monitoring approaches by integrating rapid deployment, easily configuration, real-time measurement, predictive analytics, alerts, and/or automated control. The process helps implement proactive and efficient management of water levels, prevents environmental incidents through early warning and automated response, and supports regulatory compliance through comprehensive data logging and reporting capabilities.

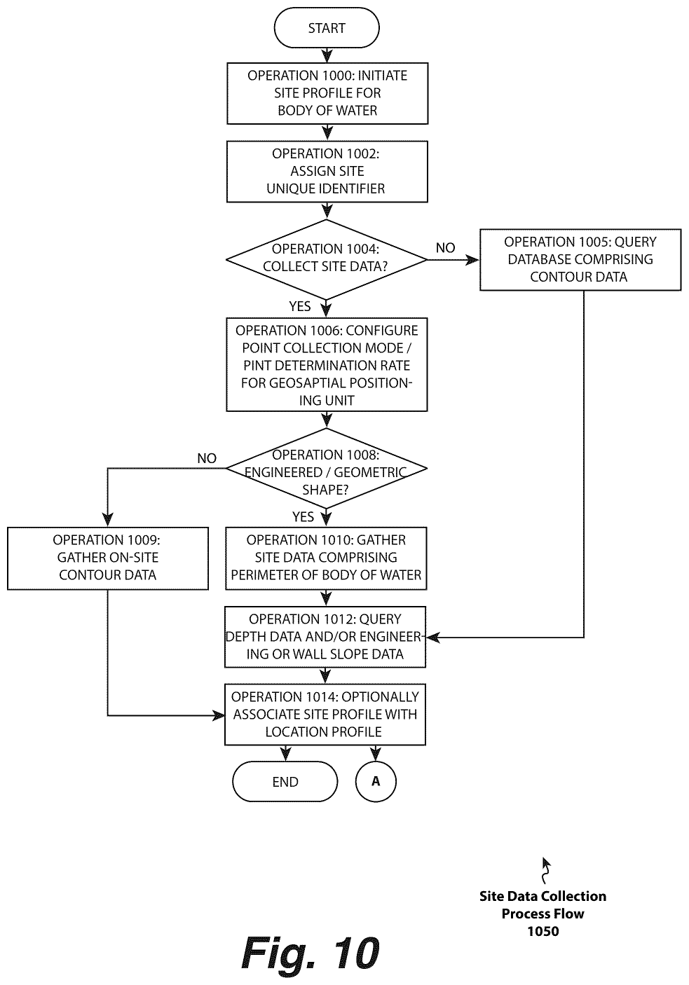

FIG. 10 illustrates a site data collection process flow 1050, according to one or more embodiments. FIG. 10 demonstrates a workflow and/or method for gathering and processing site-specific data that may be necessary to establish accurate water level monitoring capabilities for a body of water 100, e.g., gathering data usable for precise depth, volume, capacity, and/or geospatial extents calculations.

The site data collection process flow 1050 may begin with operation 1000, which may initiate a site profile 350 for the body of water 100. For example, the coordination server 300 may initiate a data object within a commercial database, e.g., an RDMS database (Postgres, Oracle®) and/or a NoSQL database (e.g., MongoDB®). Operation 1002 assigns a site unique identifier 351 to the site profile 350, providing a distinct reference for the body of water 100 and the associated site profile 350 within one or more databases.

Unique identifiers for data objects in the water monitoring system 190 (e.g., the location profile 330, the sensor profile 340, and/or the site profile 350) can utilize various approaches to ensure distinct identification across one deployed instance (or all deployed instances of) the water monitoring system 190. Universally Unique Identifiers (UUIDs) such as UUID4 may provide cryptographically strong random identifiers that reduce or eliminate collision risks when generating identifiers across multiple sensor buoys and coordination servers (e.g., a string of 32 random alphanumeric characters). In one or more other embodiments, sequential identifiers combined with device-specific prefixes (such as “BUOY001-00001” or “SITE-DAIRY-LAGOON-A-12345”) may offer human-readable formats that incorporate contextual information about the data source and sequence.

Operation 1004 may be a decision point determining whether to collect site data directly and/or query existing database information. If no site data is to be collected, for example where enough existing data exists through geospatial databases, engineered plans, and/or specifications, operation 1004 may advance to operation 1005 which may query one or more databases including contour data for the site and its embraced body of water 100. In some cases, a surface topology, especially if including elevation data, may be a complete and sufficient description of the site usable to generate the level functions 357 and perform other necessary data functions.

If site data collection is required, operation 1004 may proceed to operation 1006, which may configure the point collection mode and/or coordinate determination rate for the geospatial positioning unit 208, which may optimize data acquisition parameters for the specific site characteristics. Operation 1008 may determine whether the body of water 100 has an engineered and/or geometric shape, which may influence the data collection approach. When the site embracing the body of water 100 is engineered and/or has a relatively straightforward geometric shape, operation 1008 may proceed to operation 1010.

For engineered or geometric shapes, operation 1010 may gather site data comprising key defining geometric characteristics, such as the perimeter of the body of water 100 (e.g., the perimeter contour data 142), which may include precise boundary coordinates that define the extents and/or the containment structure of the body of water 100. For some wastewater lagoons, especially those made of earthen berms 106, surveying the berm 106 itself may be sufficient in combination with existing engineering and permit data for a full description of the shape and volume of the body of water 100.

If the body of water 100 is not engineered and/or is not describable readily through simple geometric shapes, operation 1008 may proceed to operation 1009, which may gather on-site contour data through direct measurement and/or gathering of surface contour data, for example through a user 170 walking a pattern over the area that will contain the body of water 100 (or even probing a depth of the body of water 100 at various locations). Operation 1005 and operation 1010 advance to operation 1012, which may query depth data and/or engineering or wall slope data usable for accurate volume calculations.

Operation 1014 optionally associates the site profile 350 with the location profile 330, enabling integration and relations with other site profiles 350 and/or broader environmental monitoring systems on an entire location (such as the dairy 700). The collected data provides the foundation for generating depth and volume functions that enable the coordination server 300 to accurately calculate water levels and/or capacity based on geospatial coordinates 130 received from deployed sensor buoys 200.

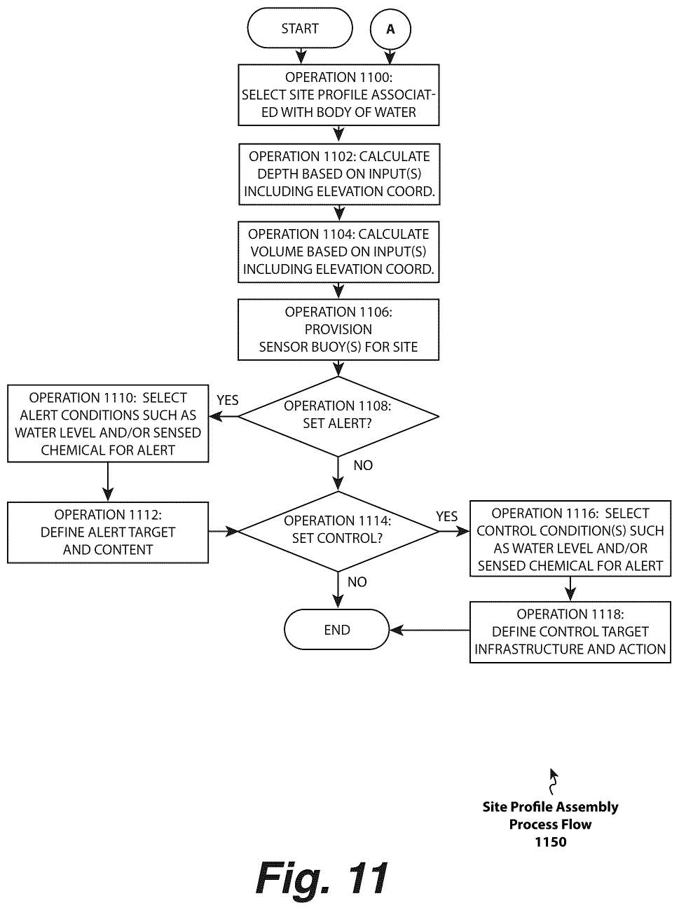

FIG. 11 illustrates a site profile assembly process flow 1150, according to one or more embodiments. FIG. 11 demonstrates a workflow and/or method for creating site profiles 350 that may enable the water monitoring system 190 to provide automated alerts 392 and/or infrastructure control based on water level.

Operation 1100 may select the site profile 350 associated with the body of water 100, for example which may have been previously initiated in a database and designated with a UID 351. Operation 1102 may calculate depth based on input that may include elevation coordinates 134, which may be used to then generate a depth function 358, according to one or more embodiments. The depth function 358 may then be stored in association with the site profile 350.

Operation 1104 may calculate volume based on input including elevation coordinates 134, for example using a dimensional representation and/or topological representation of the body of water 100, including as may be generated from the contour data 366. This information may be used to then generate a volume function 359, according to one or more embodiments, which may be stored in association with the site profile 350. Operation 1106 may provision sensor buoy(s) 200 for the site, ensuring proper deployment and/or configuration of the monitoring equipment. For example, initial data resolution, precision, and/or accuracy settings may be configured including trigger conditions or existing profiles for changes thereto.

Operation 1108 may determine whether alert conditions should be set. If alerts are to be configured, operation 1110 may select alert conditions such as water level and/or sensed chemical parameters for alert generation. Operation 1112 may then define alert target(s) (including designating communication medium) and content, for example preset messages assigned to certain water levels (e.g., a “max allowed depth alert” when the water level rises above prescribed maximum depth 116). In one or more embodiments, operation 1112 may define and store the alert profile 390 including the level 391 and alert action 393.

The method may provide automated control capabilities through decision point operation 1114 to determine whether to set control parameters. If control is enabled, operation 1116 may select control condition(s) such as water level and/or sensed chemical parameters for automated response. Operation 1118 may then define control target infrastructure (e.g., an automatic gate, a pump, a treatment device which can turn on to treat the water, etc.) and the resulting control instructions 396 to be generated. In one or more embodiments, operation 1118 may define and store the control profile 394 including the level 391 and control action 395.

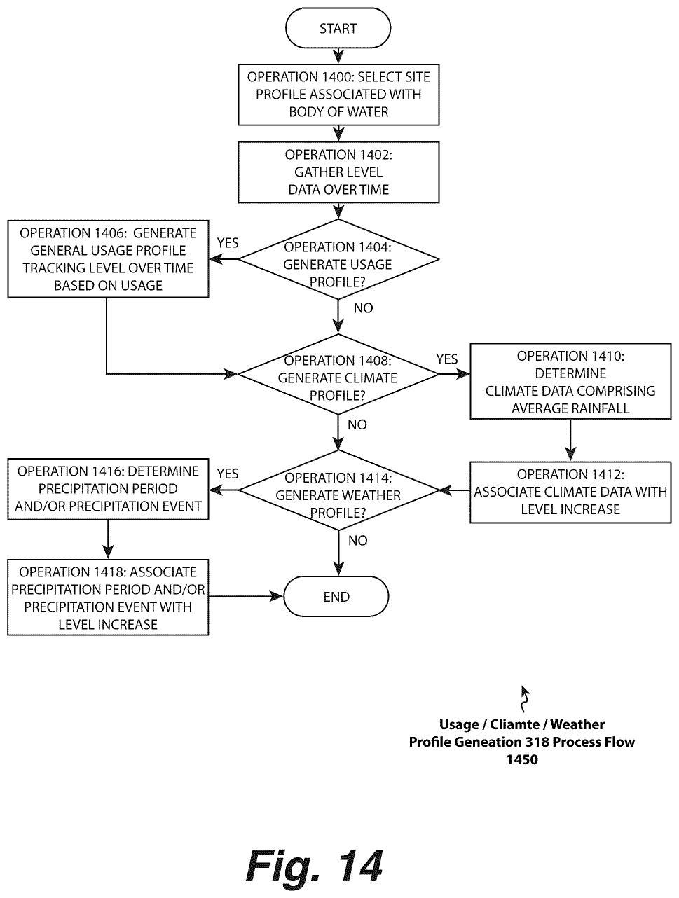

Additional aspects of generation of the site profile 350, such as those shown and described in conjunction with the embodiment of FIG. 3, also may be set up. For example, usage profiles 388, weather profiles 384, and/or climate profiles 380 may be set up as shown and described in conjunction with FIG. 3 and/or FIG. 14.

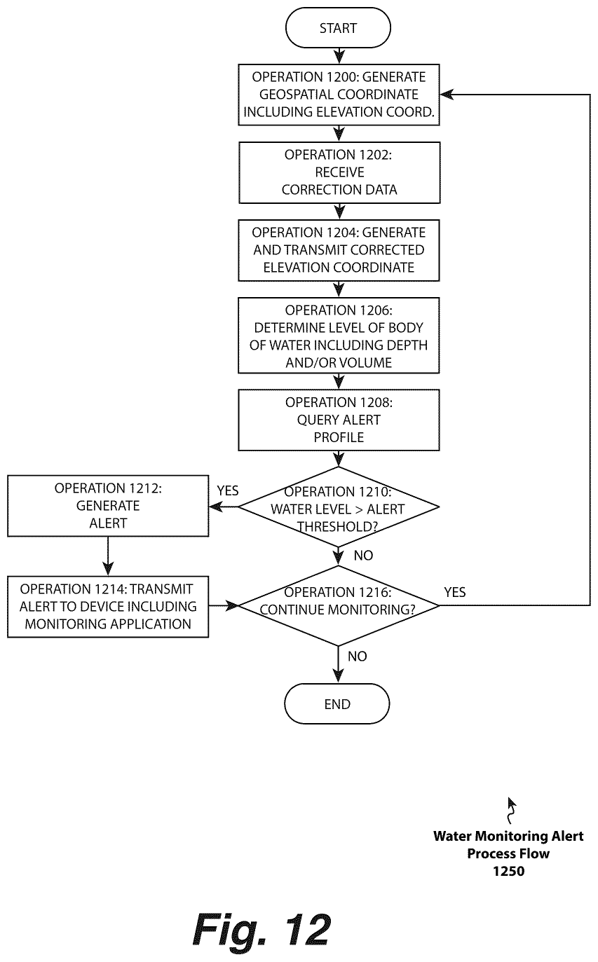

FIG. 12 illustrates a water monitoring alert process flow 1250, according to one or more embodiments. FIG. 12 illustrates a water level monitoring process and/or method that may be used to generate and transmit alerts 392 when water level conditions exceed predetermined thresholds based on use of high-precision and/or high-accuracy operation of the sensor buoy 200, helping provide early warning capabilities for overflow prevention and environmental compliance.

The water monitoring alert process flow 1250 may begin with operation 1200, which generates geospatial coordinates 130 including elevation coordinates 134 from the sensor buoy 200 positioning system. Operation 1202 may receive correction data to enhance the accuracy of the geospatial measurements (e.g., via RTK GPS or another method), followed by operation 1204 which may generate and transmit the corrected elevation coordinate 136 (and optionally other geospatial data) for processing, for example to the coordination server 300 over the network 150. It should be noted that correction may occur locally on the sensor buoy 200 and/or may occur remotely on the coordination server 300, according to one or more embodiments.

Operation 1206 may determine the level of the body of water 100, including depth and/or volume by using the corrected elevation coordinate 136. The corrected elevation coordinate 136 may be used as an input to one or more level functions 357, for example a depth function 358 and/or a volume function 359, according to one or more embodiments. Operation 1208 may then query an alert profile 390 to retrieve one or more threshold conditions and/or alert parameters for comparison to the current water level.

The process flow 1250 operation 1210 which may determine whether the current water level exceeds the configured alert threshold. If the threshold is exceeded, operation 1212 may generate an alert notification that may include relevant information about the current water level and/or potential overflow or other level-related risk. Operation 1214 may then transmit the alert 392 to a designated device (e.g., the device 400) which may include the monitoring application 402, to help ensure that operators (e.g., the user 170 and any associated organization thereof) receive immediate notification of critical conditions. In one or more other embodiments, it will be noted that the monitoring application 402 is not necessary in order to receive the alert 392, which can be sent by SMS, email, or other system accessible to the coordination server 300.

The water monitoring alert process flow 1250 may include a decision point in operation 1216 to determine whether to continue monitoring following generation of one or more alerts 392, creating a continuous loop that may enable ongoing monitoring, in which case operation 1216 may return to operation 1200. If monitoring is to cease, operation 1216 may proceed to terminate.

This water monitoring alert process 1250 provides significant advantages over traditional monitoring systems by enabling automated threshold monitoring, immediate alert 392 generation and/or transmission, and continuous surveillance capabilities based on the high-precision and/or high accuracy capabilities of the sensor buoy 200 which can be easily deployed in remote areas and areas with little or no power infrastructure.

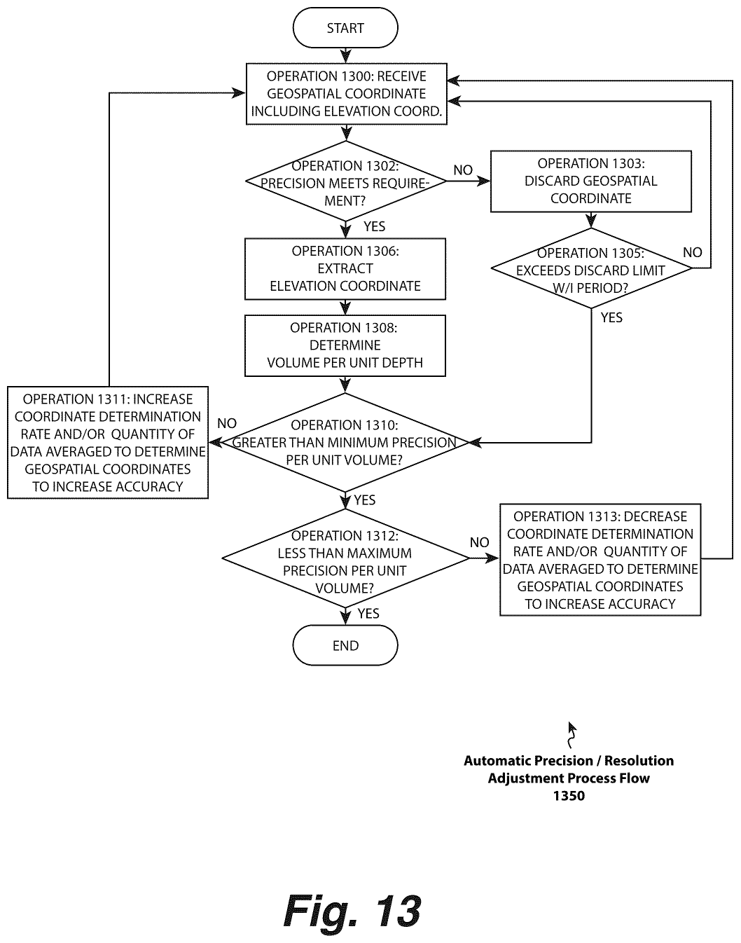

FIG. 13 illustrates data quality adjustment process flow 1350, according to one or more embodiments. FIG. 13 demonstrates dynamic optimization of measurement precision, accuracy, and/or data acquisition rates (e.g., resolution) based on current water conditions, volume characteristics, and/or other operational requirements, according to one or more embodiments.

The automatic precision and resolution adjustment process flow 1350 may begin with operation 1300, which may receive geospatial coordinates 130 including elevation coordinates 136 from the sensor buoy 200 positioning system (e.g., the geospatial positioning unit 208 and/or the spatial correction unit 211). Decision point operation 1302 may determine whether precision meets the current requirements based on water level conditions and volume per unit depth characteristics. For example, reference is made to FIG. 5 in which a data quality requirement 500 in which precision and/or data resolution may be specified as a function of depth of the body of water 100.

When data quality and/or precision requirements are not met, operation 1303 may discard the geospatial coordinate 130 to maintain data quality standards (and/or log but not otherwise act on raw data). Decision point operation 1305 may then determine whether a discard limit (e.g., two, ten, hundreds, or thousands of discarded attempts) has exceeded a limit within a specific time period. If the discard limit is exceeded, operation 1311 may increase the coordinate determination rate, coordinate resolution, and/or quantity of geospatial data averaged to determine geospatial coordinates 130, increasing probability of receiving adequate data quality appropriate for current conditions.

Operation 1306 may extract elevation coordinates 134 for processing, for example from geospatial data 260 and/or a geospatial coordinate 130 determined by the sensor buoy 200. Operation 1308 may then determine volume per unit depth based on the volume function 359, contour characteristics and/or other dimensional modeling of the body of water 100. This calculation can be important if volume changes per unit depth increase significantly as the body of water 100 approaches capacity, for example as the body of water 100 broadens out.

Operation 1310 may determine whether current conditions require greater than minimum data quality such as precision per unit volume, accuracy per unit volume, and/or greater time resolution. When higher accuracy, precision and/or resolution is needed, beneficial, or lowers the probability of missing timely detection of alert thresholds, operation 1310 may proceed to operation 1311, which may increase coordinate determination rate (e.g., coordinate resolution over time, such as more data points per unit time), and/or increase accuracy and/or precision, for example by gathering and averaging more geospatial coordinates 130.

Similarly, operation 1312 may determine whether conditions require less precision per unit volume, in which case operation 1313 may decrease the coordinate determination rate (e.g., coordinate resolution), and/or a quantity of data averaged to determine geospatial coordinates, optimizing energy efficiency while maintaining adequate monitoring capabilities.

This automatic data quality adjustment process may be advantageous for water level monitoring, including balancing measurement accuracy with energy efficiency based on actual operational needs. For example, during low-risk periods with stable water levels and minimal volume changes per unit depth, the process flow 1350 may conserve energy through reduced accuracy, precision and/or resolution requirements. During high-risk periods approaching overflow conditions where volume changes rapidly per unit depth, the system automatically increases precision and/or time resolution to ensure accurate detection and timely alert generation. This adaptive approach enables long-term autonomous operation while maintaining the high accuracy necessary for environmental compliance and overflow prevention across varying operational conditions.

FIG. 14 illustrates a usage, climate, and/or weather profile creation process flow 1450, according to one or more embodiments. Operation 1400 may select the site profile 350 associated with the body of water 100. Operation 1402 may then gather level data over time to establish baseline water level patterns and/or trends. The method may include decision point operation 1404 to determine whether to generate a usage profile 388 based on historical water level data and/or operational patterns.

When usage profile 388 is to be generated, operation 1406 may generate a general usage profile tracking level changes over time based on operational usage patterns. Usage may be determined quantitatively and/or qualitatively. For example, infrastructure including flow meters may quantitatively measure the addition of liquid to the body of water 100 in one or more industrial operations. Other quantitative measures may include indirect usage, such as a number of cows housed in a dairy served by a wastewater lagoon. In one or more other embodiments, qualitative measurements of usage may be used. For example, “heavy”, “moderate”, or “light” use may be specified based on data or user 170 experience. Provided enough data is gathered over a long enough time period, level changes attributable to usage may be isolated from effects of weather or climate through data analysis techniques known in the art of data science.

Operation 1408 may determine whether to generate a climate profile 380. If climate profiling is selected for use, operation 1408 may proceed to operation 1410 that may determine climate data comprising average rainfall and/or seasonal precipitation patterns that influence water level changes. For example, the climate data may be queried from a database that houses regional, national, and/or global climate data. Operation 1412 may then associate the climate data with level increases (or decreases) experienced, establishing correlations between precipitation periods, hot and dry periods, or other climate effects, and their corresponding water level changes. In one or more embodiments, level changes in similar nearby sites may be used to approximate or estimate climate and/or seasonal effects on the site for which the site profile 350 and the climate profile 380 is being defined.

Operation 1414 may determine whether to generate a weather profile 384 usable for real-time weather integration and prediction. When weather profiling is to be used, operation 1416 may determine past precipitation events, for example measured rainfall over a 24 hour period, measured rainfall over a single storm front, etc. Operation 1418 may then associate such precipitation events with level increases, resulting in a baseline predictive model that may predict water level increase based on weather conditions. In one or more embodiments, the sensors 240 may also include a rain gauge that may be measured and optionally emptied after each precipitation event. This integrated approach enables the system to proactively adjust data quality requirements based on expected water level changes from weather events, rather than waiting for actual level changes to occur.

It will be noted that statistical separation of water level changes attributable to usage, climate, and/or individual weather events may increase in reliability by applying a systematic approach using time series analysis and multivariate regression techniques to isolate the distinct contributions of each factor. Such analysis may begin with establishing baseline measurements over extended periods (e.g., 1 to 3 years) to capture sufficient data for each contributing factor, followed by applying decomposition methods such as seasonal-trend decomposition using LOESS (STL) or classical decomposition to separate long-term trends from seasonal patterns and short-term variations. Usage effects can be isolated by correlating water level changes with operational data such as discharge volumes, production schedules, or facility utilization rates, using techniques such as cross-correlation analysis to account for time lags between usage events and corresponding water level responses. Climate effects can be separated by analyzing long-term seasonal patterns and correlating them with historical precipitation data, temperature records, and evaporation rates using multiple linear regression models that account for seasonal coefficients and moving averages of climate variables over monthly or seasonal periods. Individual weather events are identified through residual analysis after removing usage and climate trends, where sudden water level changes that correlate with specific precipitation events, storm systems, and/or extreme weather conditions can be quantified using event-based regression models that consider precipitation intensity, duration, and antecedent conditions. The statistical separation process may employ techniques such as principal component analysis (PCA) to identify independent factors, autoregressive integrated moving average (ARIMA) modeling to account for temporal dependencies, and machine learning approaches like random forest regression to capture non-linear relationships between multiple variables. Use of such techniques may enable the coordination server 300 to generate accurate predictive models (e.g., stored within the climate profile 380, the weather profile 384, and/or the usage profile 388) that distinguish between normal operational variations, expected seasonal changes, and expected or unexpected weather events. In one or more embodiments, an artificial neural network and/or deep learning model may be used with various usage, climate, and weather inputs to predict level change as an output.

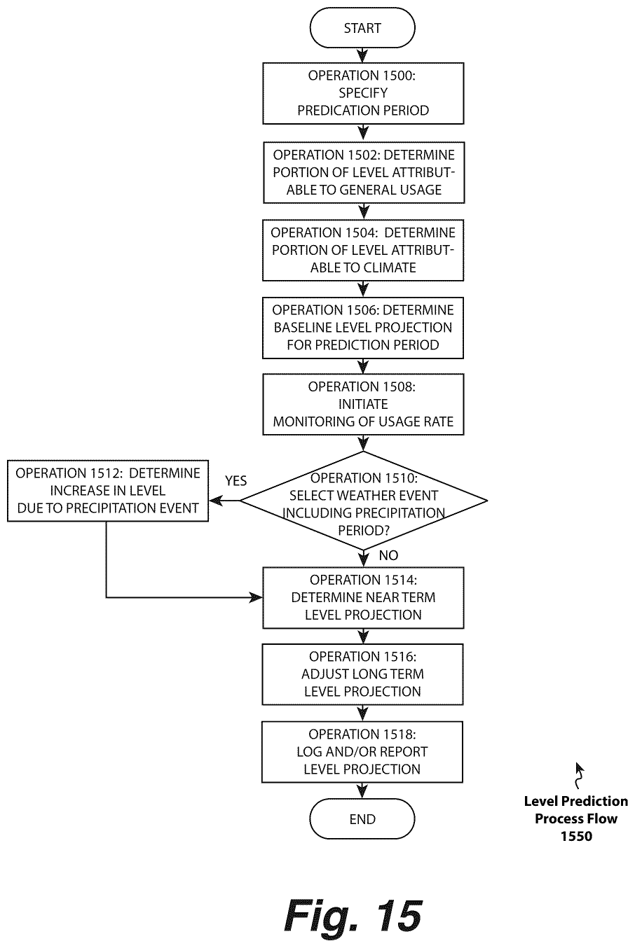

FIG. 15 illustrates a level prediction process flow 1550, according to one or more embodiments. FIG. 15 demonstrates using water level monitoring data to generate predictive analytics for future water levels, enabling proactive management and/or early warning capabilities based on usage patterns, climate data, and weather forecasts.

The level prediction process flow 1550 may initiate with operation 1500, which specifies the prediction period for the analysis (e.g., one week, one month, one year). The specified period establishes the timeframe for which water level projections may be determined. In one or more embodiments, the allowed time horizon for projection may depend on existing quality and/or quantity of data. Operation 1502 may determine the portion of level change attributable to general usage patterns, for example through use of the usage profile 388, as previously shown and described.

Operation 1504 may determine the portion of level change attributable to climate factors, incorporating seasonal precipitation patterns and long-term weather trends that influence water levels. For example, operation 1504 may reference the climate profile 380, according to one or more embodiments. Operation 1506 may then determine the baseline level projection for the specified prediction period, combining usage and/or climate factors to establish the fundamental projection model that may vary by week, month, quarter, season, and/or year.

Operation 1508 initiates monitoring of usage rates to track real-time operational changes that may affect the baseline projection. Operation 1510 may determine whether weather events including precipitation periods should be analyzed for enhanced prediction accuracy. When use of weather data is enabled, operation 1512 may determine increases in level due to precipitation events, incorporating real-time weather data and forecasts into the prediction model. For example, operation 1512 may reference the weather profile 384 to determine the expected level increase based on the forecast precipitation.

Operation 1514 determines near-term level projection based on current usage rates and/or immediate weather conditions, providing short-term predictions usable for operational planning and other purposes. It will be understood that level change attributable to usage and/or weather can be adequately accounted for in the baseline level projection. For example, where consistent usage over several weeks approximates the same rate as that which is already attributable to usage in the baseline level projection, such usage may not impact the level projection unless unusual usage exceeds what was already accounted for and/or predicted.

Operation 1516 adjusts long-term level projection by incorporating both baseline trends, recent usage, and/or anticipated weather impacts, creating comprehensive predictions that may account for what may be predominant factors in water level change. The process flow 1550 may conclude with operation 1518, which may log and/or report the level projection results. The results may also be communicated to the device 400 for viewing by the user 170.

This level prediction process may provide advantages for water level monitoring operations by enabling proactive management based on predictive analytics that accounts for usage, climate, and/or weather rather than reactive responses to current conditions. Combined with the high-accuracy and/or high-precision of the sensor buoy 200 (which may increase data quality and therefore accuracy of the climate profile 380, weather profile 384, and/or usage profile 388), the prediction may be useful for bodies of water 100 that otherwise would not be subject to predictive analysis. This predictive approach can be valuable for applications such as wastewater treatment facilities, agricultural operations, and/or industrial sites where advance knowledge of water level changes enables optimized operations and prevents environmental incidents through proactive intervention.

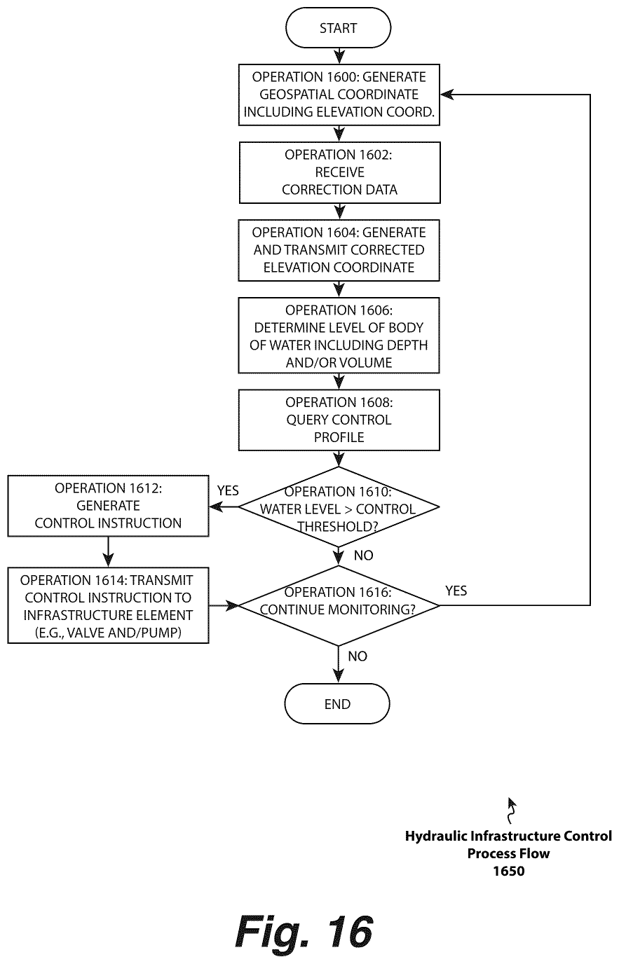

FIG. 16 illustrates a hydraulic infrastructure control process flow 1650, according to one or more embodiments. FIG. 16 demonstrates water level monitoring to automatically control hydraulic infrastructure such as valves and/or pumps based on real-time water level measurements and/or predetermined control thresholds.

The hydraulic infrastructure control process flow 1650 may initiate with operation 1600, which may generate geospatial coordinates 130 including elevation coordinates 134 from the geospatial positioning unit 208 of the sensor buoy 200. Operation 1602 may receive correction data to enhance the accuracy of the geospatial measurements, followed by operation 1604 which may generate and transmit the corrected elevation coordinate 136 to the coordination server 300 for processing. In one or more other embodiments, it will be appreciated that the elevation coordinate 136 may be used without correction and/or may be corrected on the coordination server 300 if the correction data determined by the spatial correction unit 211 is additionally transmitted to the server 300.

Operation 1606 may determine the level of the body of water 100 including both depth and/or volume using the elevation coordinate 136 as an input to one or more pre-configured site profiles 350, and specifically may use level functions 357 of the site profile 350, according to one or more embodiments. The process flow 1650 may then query the control profile 394 in operation 1608 to retrieve one or more threshold conditions and/or control parameters that may have been established for automated infrastructure management (e.g., control of the infrastructure device 160) at, or hydraulically coupled to, the specific body of water 100 being monitored.

Operation 1610 may determine whether the current water level exceeds the configured control threshold. If the threshold is exceeded, operation 1612 generates a control instruction 396 that specifies an infrastructure control and/or manipulation response, such as a valve opening, a pump activation, and/or flow redirection to prevent overflow conditions or optimize capacity utilization.

Operation 1614 transmits the control instruction 396 to the designated infrastructure device 160, such as valve controllers and/or pump systems, enabling automated response to changing water level conditions. Operation 1616 may then determine whether to continue monitoring, creating a continuous loop that enables ongoing surveillance and automated control responses as water level conditions evolve. Although valves and pumps are repeatedly described herein, it will be recognized that other infrastructure devices 160 can be used, for example water treatment equipment (e.g., chemical applicators), water agitators, water re-circulators, water aerators, etc.

Although the present embodiments have been described with reference to specific example embodiments, it will be evident that various modifications and changes may be made to these embodiments without departing from the broader spirit and scope of the various embodiments. For example, the various devices, engines, agent, routines, and modules described herein may be enabled and operated using hardware circuitry (e.g., CMOS based logic circuitry), firmware, software, or any combination of hardware, firmware, and software (e.g., embodied in a non-transitory machine-readable medium). For example, the various electrical structure and methods may be embodied using transistors, logic gates, and electrical circuits (e.g., application specific integrated circuitry (ASIC) and/or Digital Signal Processor (DSP) circuitry).

In addition, it will be appreciated that the various operations, processes, and methods disclosed herein may be embodied in a non-transitory machine-readable medium and/or a machine-accessible medium compatible with a data processing system (e.g., the infrastructure device 160, the sensor buoy 200, the controller 220, the coordination server 300, and/or the device 400). Accordingly, the specification and drawings are to be regarded in an illustrative rather than a restrictive sense.

The structures in the figures such as the engines, routines, and modules may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the preceding disclosure.

Embodiments of the invention are discussed above with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures.

From reading the present disclosure, other variations and modifications will be apparent to persons skilled in the art. Such variations and modifications may involve equivalent and other features which are already known in the art, and which may be used instead of or in addition to features already described herein.

Although claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any claim and whether or not it mitigates any or all of the same technical problems.

Features which are described in the context of separate embodiments may also be provided in combination in a single embodiment. Conversely, various features which are, for brevity, described in the context of a single embodiment, may also be provided separately or in any suitable sub-combination. The applicants hereby give notice that new claims may be formulated to such features and/or combinations of such features during the prosecution of the present application or of any further application derived therefrom.

References to “one embodiment,” “an embodiment,” “example embodiment,” “various embodiments,” “one or more embodiments,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least one or more embodiments of the invention” includes the stated particular feature, structure, or characteristic.

The enumerated listing of items does not imply that any or all of the items are mutually exclusive, unless expressly specified otherwise.

It is understood that the use of a specific component, device and/or parameter names are for example only and not meant to imply any limitations on the invention. The invention may thus be implemented with different nomenclature and/or terminology utilized to describe the mechanisms, units, structures, components, devices, parameters and/or elements herein, without limitation. Each term utilized herein is to be given its broadest interpretation given the context in which that term is utilized.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; a smartphone, application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, one or more embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hypertext Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

The term “processor” may refer to any device or portion of a device that processes electronic data from registers and/or memory to transform that electronic data into other electronic data that may be stored in registers and/or memory. A “computing platform” may comprise one or more processors.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the water monitoring system 190 and components thereof according to the present invention will be apparent to those skilled in the art. Various aspects of the invention have been described above by way of illustration, and the specific embodiments disclosed are not intended to limit the invention to the particular forms disclosed. The particular implementation of the water monitoring system 190 may vary depending upon the particular context or application. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A sensor buoy for monitoring water level of a body of water, the sensor buoy comprising:
a float for floating the sensor buoy on a surface of the body of water,
an elevation rod coupled to the float,
a geospatial positioning unit coupled to the elevation rod and electrically coupled to a power source,
wherein the power source comprising a battery coupled to the sensor buoy, and
wherein the geospatial positioning unit comprises a GPS unit and a spatial correction chip comprising an RTK unit;
an energy generator coupled to the sensor buoy,
wherein the energy generator comprising a solar panel,
a wireless network interface controller configured to communicatively couple to a server through a wireless network,
a processor,
a computer readable media that is non-transitory comprising computer readable instructions that when executed:
determine a first geospatial coordinate and a first precision value from the geospatial positioning unit;
receive a correction data from the spatial correction chip;
generate a corrected geospatial coordinate; and
transmit the corrected geospatial coordinate to the server over the wireless network;
a tether coupled to the float, and
an anchor coupled to the tether for anchoring the sensor buoy to an anchor point on a floor of the body of water.

2. The sensor buoy of claim 1, wherein the geospatial positioning unit is coupled to the elevation rod at a first end of the elevation rod and the elevation rod is coupled to the float at a second end of the elevation rod, the sensor buoy further comprising:
a rod coupler configured to detachably couple the elevation rod to the float at the first end of the elevation rod such that the elevation rod usable as a survey rod to easily gather a site data for the body of water.

3. The sensor buoy of claim 1, wherein the computer readable media further comprising computer readable instructions that when executed:
receive a request to initiate a site acquisition mode to gather a site data for the body of water;
configure at least one of (i) a continuous point acquisition mode determining geospatial coordinates at a first coordinate determination rate at least as fast as one point per ten seconds and (ii) a manual point acquisition mode,
initiate a site data object for the body of water;
gather the site data comprising a first set of geospatial coordinates, each geospatial coordinate of the first set of geospatial coordinates paired with a precision value;
receive a request to end the site acquisition mode gathering the site data for the body of water;
commit the site data to the site data object; and
transmit the site data object to the server over the wireless network.

4. The sensor buoy of claim 1, wherein the computer readable media further comprising computer readable instructions that when executed:
receive a reduced data quality request in response to a drop in a depth of the body of water, and
configure a second coordinate determination rate that at lest one of (i) slows the rate at which geospatial coordinates are determined to increase an energy efficiency of the power source; and (ii) reduces a quantity of geospatial coordinates gathered for calculating average geospatial coordinates.

5. The sensor buoy of claim 1, wherein the computer readable media further comprising computer readable instructions that when executed:
set a timer;
initiate a low power mode;
determine expiration of the up timer;
initiate an active mode; and
determine the first geospatial coordinate and the first precision value from the geospatial positioning unit upon entering the active mode to increase an energy efficiency of the power source.

6. The sensor buoy of claim 1, further comprising:
a ballast coupled to the float to weight the sensor buoy such that the elevation rod remains upright when the sensor buoy floats on the body of water;
an environmental sensor comprising at least one of a humidity sensor, a temperature sensor, and a wind sensor;
a water quality sensor comprising at least one of an oxygenation sensor, a nitrate sensor, a phosphate sensor, a pathogen sensor, and a heavy metal sensor; and
a backup battery,
wherein the tether comprises a corrosion resistant material configured to resist a corrosive chemical in the body of water, and
wherein the corrosion resistant material comprises at least one of stainless steel and an organic polymer.

7. A system for monitoring water level in a body of water, the system comprising:
a network;
a sensor buoy communicatively coupled to the network, comprising:
a geospatial positioning unit coupled to the elevation rod and electrically coupled to a power source,
a wireless network interface controller configured to communicatively couple to a server through a wireless network,
a processor of the sensor buoy
a computer readable media of the sensor buoy that is non-transitory comprising computer readable instructions that when executed:
determine a first geospatial coordinate comprising an elevation coordinate and a first precision value from the geospatial positioning unit;
receive a correction data from the spatial correction chip;
generate a corrected geospatial coordinate; and
transmit the corrected geospatial coordinate;
a server computer comprising:
a process of the server computer; and
a computer readable media of the server computer that is non-transitory comprising computer readable instructions that when executed:
receive at least one of (i) the first geospatial coordinate and the correction data and (ii) the corrected geospatial coordinate from the sensor buoy;
query a site profile of the body of water;
determine a level of the body of water comprising at least one of:
(i) inputting the elevation coordinate into a depth function for the wastewater lagoon and determining a depth of wastewater in the wastewater lagoon, and
(ii) inputting the depth of the wastewater into a volume function of the wastewater lagoon generated based on a contour map of the wastewater lagoon and determining a volume of the wastewater in the wastewater lagoon;
determine at least one of (i) the depth of wastewater lagoon exceeds a threshold depth, and (ii) the volume of the wastewater lagoon exceeds a threshold volume; and
generate a potential overflow alert that the wastewater lagoon exceeds at least one of the threshold depth and the volume threshold.

8. The system of claim 7, wherein the memory of the sensor buoy further comprising computer readable instructions that when executed:
receive a request to initiate a site acquisition mode to gather a site data for the body of water;
configure at least one of (i) a continuous point acquisition mode determining geospatial coordinates at a first coordinate determination rate at least as fast as one point per ten seconds and (ii) a manual point acquisition mode,
initiate a site data object for the body of water;
gather the site data comprising a first set of geospatial coordinates, each geospatial coordinate of the first set of geospatial coordinates paired with a precision value;
receive a request to end the site acquisition mode gathering the site data for the body of water;
commit the site data to the site data object;
transmit the site data object to the server over the wireless network;
receive a reduced data quality requirement request in response to a drop in a depth of the body of water;
configure a second coordinate determination rate that at lest one of (i) slows the rate at which geospatial coordinates are determined to increase an energy efficiency of the power source; and (ii) reduces a quantity of geospatial coordinates gathered for calculating average geospatial coordinates;
set a timer;
initiate a low power mode;
determine expiration of the up timer;
initiate an active mode; and
determine the first geospatial coordinate and the first precision value from the geospatial positioning unit upon entering the active mode to increase an energy efficiency of the power source.

9. The system of claim 7, wherein the memory of the server further comprising computer readable instructions that when executed:
read the precision value upon receipt of the corrected geospatial coordinate;
determine the precision value does not meet a precision requirement;
delete the precision value;
optionally increase a coordinate determination rate of the sensor buoy;
store a water level data comprising at least one of an elevation value of wastewater in the wastewater lagoon over time, a depth of the wastewater in the wastewater lagoon over time, and a volume of the wastewater in the wastewater lagoon over time, generate a level projection for the wastewater lagoon is based on inputs comprising the wastewater level data;
determine a date in which a remaining capacity of the wastewater lagoon is exceeded.
generate a climate profile comprising average rainfall;
associate a precipitation period with an increase in wastewater level;
estimate an increase in the wastewater level based in the climate profile,
wherein the level projection for the wastewater lagoon is based on inputs further comprising the increase in the wastewater level based on the climate profile;
determine occurrence of a precipitation event;
associate the precipitation event with an increase in the wastewater level of the wastewater lagoon;
receive weather forecast data;
estimate an increase in the water level based in the weather forecast data,
wherein the level projection for the wastewater lagoon is based on inputs further comprising the increase in the wastewater level based on the weather forecast data;
increase at least one of a precision and the coordinate determination rate in response to an increase in level of the wastewater to account for increase volume per unit depths as the body of water fills;

10. The system of claim 7, further comprising:
a second sensor buoy in a second body of water at least one of hydrologically coupled and hydraulicly coupled to the first body of water;
wherein the memory of the server further comprising computer readable instructions that when executed:
receive a second geospatial coordinate from the second sensor buoy comprising an elevation coordinate of the second geospatial coordinate;
determine a wastewater depth of the second wastewater lagoon;
determine that the second wastewater lagoon has a remaining capacity to accept discharge from the wastewater lagoon;
generate a control instruction comprising at least one of a valve control instruction and a pump control instruction; and
transmit the control instruction to at least one of a valve controller and a pump controller through a network to automatically initiate flow of water from the body of water to the second body of water.

11. The system of claim 10 further comprising:
a device communicatively coupled to the server through the network, comprising:
a processor of the device;
a memory of the device that is a non-transitory computer readable memory comprising a monitoring application comprising computer readable instructions that when executed:
receive the potential overflow alert to a device comprising a monitoring application, and
wherein the memory of the server further comprising computer readable instructions that when executed transmit the potential overflow alert to the device.

12. The system of claim 11,
wherein the geospatial positioning unit comprises a GPS unit and a spatial correction chip comprising an RTK unit,
wherein the first coordinate determination rate is at least as fast as one point per ten seconds.

13. A method for monitoring water level in a wastewater lagoon, the method comprising:
generating a first geospatial coordinate at a sensor buoy comprising a geospatial positioning unit,
wherein the first geospatial coordinate comprises an elevation coordinate;
receiving a correction data at the sensor buoy and correcting the first geospatial coordinate with the correction data to generate a corrected geospatial coordinate,
wherein the corrected geospatial coordinate comprising a precision value following correction by the correction data;
querying a site profile of the wastewater lagoon;
inputting the elevation coordinate into a depth function for the wastewater lagoon;
determining a depth of wastewater in the wastewater lagoon;
inputting the depth of the wastewater into a volume function of the wastewater lagoon generated based on a contour map of the wastewater lagoon;
determining a volume of the wastewater in the wastewater lagoon;
determining at least one of (i) the depth of wastewater lagoon exceeds a threshold depth, and (ii) the volume of the wastewater lagoon exceeds a threshold volume;
generating a potential overflow alert that the wastewater lagoon exceeds at least one of the threshold depth and the volume threshold; and
transmitting the potential overflow alert to a device comprising a monitoring application.

14. The method of claim 13, further comprising:
receive a request to initiate a site acquisition mode to gather a site data for the wastewater lagoon;
configure a first coordinate determination rate,
initiate a site profile for the wastewater lagoon;
gather the site data comprising a first set of geospatial coordinates collected as the geospatial positioning unit of the sensor buoy travels at least one of in and around the wastewater lagoon;
receive a request to end the site acquisition mode gathering the site data for the wastewater lagoon;
receiving the site data at a coordination server;
generating a site polygon by bounding the first set of geospatial coordinates;
referencing a maximum depth value of the wastewater lagoon;
determining a slope specification of the wastewater lagoon;
generating at least one of a depth function for the wastewater lagoon and a volume function of the wastewater lagoon; and
generating a site profile of the wastewater lagoon and associating at least one of the volume function of the wastewater lagoon and the depth function of the wastewater lagoon.

15. The method of claim 14, further comprising:
reading the precision value upon receipt of the corrected geospatial coordinate;
determining the precision value does not meet a precision requirement;
deleting the precision value; and
optionally increasing a coordinate determination rate of the sensor buoy.

16. The method of claim 15, further comprising:
storing a wastewater level data comprising at least one of an elevation coordinate of wastewater in the wastewater lagoon over time, a depth of the wastewater in the wastewater lagoon over time, and a volume of the wastewater in the wastewater lagoon over time,
generating a level projection for the wastewater lagoon is based on inputs comprising the wastewater level data;
determining a date in which a remaining capacity of the wastewater lagoon is exceeded.
generating a climate profile comprising average rainfall;
associating a precipitation period with an increase in wastewater level;
estimating an increase in the wastewater level based in the climate profile,
wherein the level projection for the wastewater lagoon is based on inputs further comprising the increase in the wastewater level based on the climate profile;
determining occurrence of a precipitation event;
associating the precipitation event with an increase in the wastewater level of the wastewater lagoon;
receiving a weather forecast data; and
estimating an increase in the wastewater level based in the weather forecast data,
wherein the level projection for the wastewater lagoon is based on inputs further comprising the increase in the wastewater level based on the weather forecast data.

17. The method of claim 16, further comprising:
generating a control instruction comprising at least one of a valve control instruction and a pump control instruction; and
transmitting the control instruction to at least one of a valve controller and a pump controller through a network.

18. The method of claim 17, further comprising:
increasing at least one of a precision and the coordinate determination rate in response to an increase in elevation of the wastewater to account for increase volume per unit depths as the wastewater lagoon fills.

19. The method of claim 18, further comprising:
generating a second geospatial coordinate at a second sensor buoy in a second wastewater lagoon,
wherein the second geospatial coordinate of the second wastewater lagoon comprising an elevation coordinate of the second geospatial coordinate;
determining a wastewater depth of the second wastewater lagoon;
determining that the second wastewater lagoon has a remaining capacity to accept discharge from the wastewater lagoon; and
automatically initiating flow of wastewater from the wastewater lagoon to the second wastewater lagoon.

20. The method of claim 19,
wherein the first coordinate determination rate is at least as fast as one point per ten seconds.
